(19) United States
(12) Patent Application Publication
(10) Pub. No.: US 2022/0336043 A1
(43) Pub. Date: Oct. 20, 2022

(54) cfDNA CLASSIFICATION METHOD, APPARATUS AND APPLICATION

(71) Applicants: Beijing Institute of Genomics,Chinese Academy of Sciences (China National Center for Bioinformation), Beijing (CN); Peking University First Hospital, Beijing (CN)

(72) Inventors: Weimin CI, Beijing (CN); Guangzhe GE, Beijing (CN); Yuanyuan ZHOU, Beijing (CN); Xuesong LI, Beijing (CN)

(21) Appl. No.: 17/609,036

(22) PCT Filed: Apr. 29, 2020
(86) PCT No.: PCT/CN2020/087830

§ 371 Date: Apr. 5, 2022

(30) Foreign Application Priority Data
May 7, 2019 (CN) 201910374094.1

(51) Int. Cl. G16B 20/00; C12Q 1/6886; G16B 30/00; G16B 40/20; G16H 50/70; G16H 70/60

(52) U.S. Cl. CPC G16B 20/00; C12Q 1/6886; G16B 30/00; G16B 40/20; G16H 50/70; G16H 70/60; C12Q 1/6869

(57) ABSTRACT

The invention pertains to the field of genomics and bioinformatics, and relates to a cfDNA classification method, apparatus and application. Specifically, the present invention relates to a cfDNA classification method, comprising: calculating a copy number variation data of cfDNA in a target sample; calculating a similarity degree between the target cfDNA copy number variation data and the cfDNA copy number variation data of each category label; and determining the category to which the target cfDNA belongs according to the similarity degree by using a classifier model. The invention can realize the diagnosis of up to 3 types of urogenital system tumors at one time, and has high sensitivity and specificity. In particular, in the diagnosis and dynamic monitoring of urothelial cancer, the sensitivity and specificity are higher than those of the current clinical detection methods.

### CROSS-REFERENCE TO RELATED APPLICATIONS

This patent application is the U.S. National Stage of International Patent Application No. PCT/CN2020/087830, filed Apr. 29, 2020, which claims priority to Chinese Patent Application No. 201910374094.1, filed May 7, 2019, each of which is hereby incorporated by reference in its entirety.

### FIELD OF THE INVENTION

The invention pertains to the field of genomics and bioinformatics, and relates to a cfDNA classification method, apparatus and application.

### BACKGROUND OF THE INVENTION

Urogenital system tumors (prostate cancer, urothelial cancer and renal cancer) are serious diseases that endanger human health. The diagnosis and monitoring methods for urogenital system tumors are usually invasive, or lack sensitivity and specificity.

Renal cancer accounts for about 3% of adult malignant tumors and 90% to 95% of kidney tumors, of which about 75% are renal clear cell carcinomas. At present, surgical treatment is still the most effective treatment for localized renal cancer, but about 20% to 40% of patients will suffer the relapse after surgery. Renal cell carcinoma has low sensitivity to radiotherapy and chemotherapy. The mortality rate of renal cancer patients is as high as 40%. The high mortality rate caused by renal cancer is mainly due to the lack of obvious clinical symptoms in the early stage and the lack of effective treatment methods in the advanced stage. At present, imaging, fine needle aspiration (FNA), and core biopsy (CB) can only assist in monitoring and cannot give a clear diagnosis. At present, there is no tumor marker with good sensitivity and specificity that can be used for early diagnosis and postoperative follow-up of renal cancer.

Urothelial carcinoma is a malignant tumor that occurs in renal pelvis, ureter, bladder, urethra, etc. and covers transitional epithelial cells. It mainly includes upper urothelial cancer and bladder cancer where the renal pelvis and ureter are located. Among them, upper urothelial cancer is relatively rare, accounting for only 5% to 10% of urothelial cancers, but in China, the upper urothelial cancer accounts for a proportion of as high as 30% of urothelial cancers. A number of studies have shown that the regional characteristics of upper urothelial cancer may be related to the use of traditional Chinese medicine containing aristolochic acid and its analogues. In addition, although the tissue sources are the same, upper urothelial cancer and bladder cancer have very different clinicopathological characteristics. Screening of new risk factors, new targets, and new markers for diagnosis, prognosis and dynamic monitoring of urothelial cancer must consider these two subtypes of cancer at the same time. In addition, the high recurrence rate of urothelial cancer in patients may lead to an increase in number of operations, an increase in incidence of complications, and an increase in treatment costs. Patients with recurrence eventually need to undergo radical cystectomy or bilateral nephroureterectomy, which greatly reduces the survival rate and quality of life. At present, the diagnosis of bladder cancer can be performed by the imaging, fluorescence in situ hybridization FISH, and urine cytology auxiliary examination, but the sensitivity for low-grade bladder tumors is only 4% to 31%. At present, the most important method for diagnosing bladder cancer is cystoscopy, but cystoscopy is expensive and invasive, which increases the patient's pain. In addition, the recurrence rate of bladder cancer is high, and cystoscopy is inconvenient for long-term, lifelong and prognostic monitoring.

Prostate cancer is a common malignant tumor in men, and the incidence is on the rise to a certain extent. There are no symptoms in the early stage of prostate cancer. When the tumor develops to a certain extent, it will block urethra or invade bladder neck, causing frequent urination, urinary urgency, and urinary incontinence. Many patients are already in the advanced stage when a definite diagnosis is made, and many patients in the advanced stage have bone metastases. At present, the accepted diagnostic methods for prostate cancer are digital rectal examination and prostate-specific antigen (PSA) examination, but the level of PSA can also be affected by factors such as prostatitis, urinary retention, catheterization and drugs, resulting in a lot of false positive rates.

With the development of science and technology, the diagnosis technology for tumors is also constantly advancing. In June 2017, the World Economic Forum and the Expert Committee of Scientific American jointly selected the 2017 global top ten emerging technologies list, among which the non-invasive diagnostic technology for tumors was successfully selected and ranked first. The emergence of tumor non-invasive diagnostic technology, i.e., liquid biopsies, marks another big step forward for human beings on the road of conquering tumors. Compared with traditional tissue biopsy, liquid biopsy has unique advantages such as real-time dynamic detection, overcoming tumor heterogeneity, and providing comprehensive detection information. At present, in clinical research, liquid biopsy mainly includes free circulating tumor cells (CTCs) detection, circulating tumor DNA (ctDNA) detection, exosomes and circulating RNA (Circulating RNA) detection, etc.; as compared with traditional diagnostic technology relying on clinical symptoms or imaging, the use of liquid biopsy technology can detect disease progression earlier. Liquid biopsy is expected to play a major role in evaluating tumor dynamics and load changes during patient treatment, monitoring the effectiveness of treatment in real time, and monitoring small residual lesions, recurrence, prognostic evaluation, and drug resistance in patients.

At present, there is still a need to develop new detection methods for urogenital system tumors, which have better specificity and sensitivity, are more convenient for multiple, long-term and prognostic monitoring, and reduce patient suffering.

### BRIEF SUMMARY OF THE INVENTION

After in-depth research and creative work, the present inventors surprisingly found that the detection of free DNA (cfDNA) in urine supernatant is beneficial to the detection or diagnosis of an early stage, low-grade, non-invasive tumor in urinary system. Furthermore, the present inventors designed and completed experiments, sequencing and analysis, and by detecting the cfDNA copy number variation (CNV) in the urine supernatant, the diagnosis and classification of up to 3 urogenital system tumors can be completed at one time. The following invention is therefore provided:

### BRIEF DESCRIPTION OF THE SEVERAL VIEWS OF THE DRAWINGS

FIG. 1: classification results of random forest binary classifier for renal cancer vs. normal: sensitivity 72.2%, specificity 93.1%, accuracy rate 85.1%.

FIG. 2: classification results of random forest binary classifier for urothelial carcinoma vs. normal: sensitivity 76.2%, specificity 100%, accuracy rate 90.0%.

FIG. 3: classification results random of forest binary classifier for prostate cancer vs. normal: sensitivity 71.4%, specificity 93.1%, accuracy rate 86.1%.

FIG. 4: classification results of random forest binary classifier for renal cancer vs. prostate cancer: sensitivity 72.2%, specificity 85.7%, accuracy rate 78.1%.

FIG. 5: classification results random of random forest binary classifier for urothelial cancer vs. renal cancer: sensitivity 95.2%, specificity 77.8%, accuracy rate 87.2%.

FIG. 6: classification results random of random forest binary classifier for urothelial cancer vs. prostate cancer: sensitivity 85.7%, specificity 85.7%, accuracy rate 85.7%.

FIG. 7A shows a schematic diagram of the GUdetector integrated classification model.

FIG. 7B shows the classification results of the integrated classification decision-making system (GUdetector) in four categories, the prediction accuracy of each category was 89.7% for the normal group, 76.2% for urothelial cancer, 64.3% for prostate cancer, and 44.4% for renal cancer, and the overall accuracy rate was 72.0%.

FIG. 8 shows the diagnosis model of prostate cancer in male sample. For prostate cancer vs. normal: the accuracy rate was 96.7%.

FIG. 9 shows the SVM classification results (considering gender factors and removing markers on all sex chromosomes) in four categories, the prediction accuracy rate of each category was 84.7% for the normal group, 74.3% for urothelial cancer, 52.2% for prostate cancer, and 55.8% for renal cancer, the overall accuracy rate was 70.1%.

FIG. 10 shows the SVM classification results in three categories, and the prediction accuracy rate was 88.5% for the normal group, 76.1% for urothelial cancer, 64.8% for renal cancer, and the overall accuracy rate was 78.4%.

FIG. 11 shows the SVM classification results of urothelial carcinoma (defined as UCdetector), and the comparison with LASSO and random forest methods. For the SVM, the prediction accuracy rate was 94.7% for the normal group, 86.5% for urothelial cancer, and the overall accuracy rate was 91.4%. For the LASSO, the prediction accuracy was 94.7% for the normal group, 75.0% for urothelial carcinoma, and the overall accuracy rate was 86.72%. For the random forest method, the prediction accuracy was 97.4% for the normal group, 80.8% for urothelial cancer, and the overall accuracy rate was 89.8%.

FIGS. 12A to 12D show the examples of dynamic monitoring of therapeutic efficacy of urothelial cancer, wherein:

FIG. 12A shows the postoperative dynamic monitoring of Patient 1;

FIG. 12B shows the postoperative dynamic monitoring of Patient 2;

FIG. 12C shows the postoperative dynamic monitoring of Patient 3; and

FIG. 12D shows the summary of postoperative dynamic monitoring of 3 patients.

### DETAILED DESCRIPTION OF THE INVENTION

One aspect of the present invention relates to a cfDNA classification method, comprising:

calculating a copy number variation data of cfDNA in a target sample;

calculating a similarity degree between the target cfDNA copy number variation data and the cfDNA copy number variation data of each category label; and

determining the category to which the target cfDNA belongs by using a classifier model according to the similarity degree.

In some embodiments of the present invention, in the classification method, to determine the category to which the target cfDNA belongs comprises:

according to the similarity degree, using a random forest model to determine the correlation degree between the cfDNA copy number variation data of each category label and a human urogenital system tumor;

according to the correlation degree, using the classifier model to determine the category to which the target cfDNA belongs.

In some embodiments of the present invention, in the classification method, to determine the correlation degree between the cfDNA copy number variation data of each category label and the human urogenital system tumor comprises:

according to the correlation degree, sorting the cfDNA copy number variation data to form a vector sequence;

inputting the vector sequence into the random forest model, and determining a correlation degree between the cfDNA copy number variation data of the category label and the human urogenital system tumor.

In some embodiments of the present invention, in the classification method, the human urogenital system tumor is one or more selected from the group consisting of prostate cancer, urothelial cancer and renal cancer;

preferably, the renal cancer is clear renal cell carcinoma,

preferably, the urothelial cancer is upper urothelial cancer and/or bladder cancer,

preferably, the prostate cancer is prostate adenocarcinoma;

preferably, the human urogenital system tumor is diagnosed by tissue biopsy of a surgical sample.

In some embodiments of the present invention, in the classification method, the random forest model is at least 3 random forest binary classifiers, and is one, two, three or four groups selected from the group consisting of the following Groups I to VI:

Group I.

normal-vs-renal cancer, normal-vs-urothelial cancer, normal-vs-prostate cancer;

Group II.

renal cancer-vs-normal, renal cancer-vs-urothelial cancer, renal cancer-vs-prostate cancer;

Group III.

urothelial cancer-vs-normal, urothelial cancer-vs-renal cancer, urothelial cancer-vs-prostate cancer;

Group IV.

prostate cancer-vs-normal, prostate cancer-vs-renal cancer, prostate cancer-vs-urothelial cancer.

In some embodiments of the present invention, in the classification method, each group is voted, the category corresponding to the group with the highest number of votes is the final category, and if there are groups with the same number of votes, the category corresponding to the group with the highest prediction probability in the groups with the same number of votes is the final category, and the present inventors define this integrated classification method as GUdetector.

In some embodiments of the present invention, in the classification method, the copy number variation data of cfDNA in the target sample and/or the cfDNA copy number variation data of each category label is obtained by calculation from a sequencing data of cfDNA in a urine sample; preferably, the sequencing data is a whole-genome sequencing data; preferably, its sequencing depth is 1× to 5×.

In some embodiments of the present invention, in the classification method, the copy number variation data of cfDNA in the target sample and/or the cfDNA copy number variation data of each category label is calculated according to the following method:

dividing a genome of a sample to be tested into 5,000 to 500,000 bins (for example, 50,000 bins) with equal lengths or equal theoretical simulation copy numbers; normalizing the sequencing data, and calculating a ratio A/B of the number of reads corresponding to each bin,

wherein:

A represents the actual number of reads in a bin after GC content correction;

B represents the theoretical number of reads in the bin, is obtained by dividing the total number of reads measured in the sample by the total number of bins;

the ratio A/B represents the copy number variation.

In one or more embodiments of the present invention, in the classification method, the genome of the sample to be tested is divided into 5,000 to 500,000 bins with equal lengths or equal theoretical simulation copy numbers by a software or algorithm, such as Varbin, CNVnator, ReadDepth or SegSeq.

In one or more embodiments of the present invention, in the classification method, the ratio A/B of the number of reads corresponding to each bin is calculated by a software or algorithm, such as Varbin, CNVnator, ReadDepth, or SegSeq.

In one or more embodiments of the present invention, in the classification method, the genome of the sample to be tested is divided into 10,000 to 200,000 bins with equal lengths or equal theoretical simulation copy numbers.

In one or more embodiments of the present invention, in the classification method, the genome of the sample to be tested is divided into 10,000 to 150,000 bins with equal lengths or equal theoretical simulation copy numbers.

In one or more embodiments of the present invention, in the classification method, the genome of the sample to be tested is divided into 10,000 to 100,000 (for example, 10,000, 20,000, 30,000, 40,000, 50,000, 60,000, 70,000, 80,000, 90,000 or 100000) bins with equal lengths or equal theoretical simulation copy numbers.

In some embodiments of the present invention, in the classification method, the urine sample is a morning urine; preferably, the urine sample is a morning urine supernatant.

In some embodiments of the present invention, in the classification method, the ratio A/B is a ratio A/B of each biomarker in a biomarker combination,

wherein,

the biomarker combination is any one of the biomarker combinations of the present invention described below.

Another aspect of the present invention relates to a method for the detection, diagnosis, classification, disease risk assessment or prognosis assessment of a human urogenital system tumor, which comprises the following step (1), step (2), optionally step (3), and step (4):

(1) collecting a urine sample and extracting cfDNA;

(2) screening to obtain cfDNA fragments of 90 to 300 bp or cfDNA fragments of 100 to 300 bp,

(3) using the obtained cfDNA fragments to construct a whole-genome library; preferably, performing whole-genome sequencing on the whole-genome library; and

(4) classifying the cfDNA fragments by the classification method according to any one of items of the present invention. The cfDNA fragments are the cfDNA fragments obtained in step (2) or the cfDNA fragments in the whole genome library in step (3).

In some embodiments of the present invention, in the method, the human urogenital system tumor is one or more selected from the group consisting of prostate cancer, urothelial cancer and renal cancer;

preferably, the renal cancer is clear renal cell carcinoma,

preferably, the urothelial cancer is upper urothelial cancer and/or bladder cancer,

preferably, the prostate cancer is prostate adenocarcinoma.

In some embodiments of the present invention, in the method, in step (1), the urine sample is a morning urine; preferably, the urine sample is a morning urine supernatant.

In some embodiments of the present invention, in the method, in step (2), the screening is a magnetic bead screening.

Another aspect of the present invention relates to an apparatus for the detection, diagnosis, classification, disease risk assessment or prognosis assessment of a human urogenital system tumor, comprising:

I. ‘normal decision-making unit’:

normal-vs-renal cancer, normal-vs-urothelial cancer, normal-vs-prostate cancer;

II. ‘renal cancer decision-making unit’:

renal cancer-vs-normal, renal cancer-vs-urothelial cancer, renal cancer-vs-prostate cancer;

III. ‘urothelial cancer decision-making unit’:

urothelial cancer-vs-normal, urothelial cancer-vs-renal cancer, urothelial cancer-vs-prostate cancer; and

IV. ‘prostate cancer decision-making unit’:

prostate cancer-vs-normal, prostate cancer-vs-renal cancer, prostate cancer-vs-urothelial cancer.

Another aspect of the present invention relates to an apparatus for the detection, diagnosis, classification, disease risk assessment or prognosis assessment of a human urogenital system tumor,

comprising a memory; and a processor coupled to the memory,

wherein,

the memory stores a program instruction to be executed by a processor, and the program instruction comprises any one, any two, any three, or all of four decision-making units selected from the group consisting of the following four decision-making units, wherein each decision-making unit comprises 3 random forest binary classifiers:

I. ‘normal decision-making unit’:

normal-vs-renal cancer, normal-vs-urothelial cancer, normal-vs-prostate cancer;

II. ‘renal cancer decision-making unit’:

renal cancer-vs-normal, renal cancer-vs-urothelial cancer, renal cancer-vs-prostate cancer;

III. ‘urothelial cancer decision-making unit’:

urothelial cancer-vs-normal, urothelial cancer-vs-renal cancer, urothelial cancer-vs-prostate cancer;

IV. ‘prostate cancer decision-making unit’:

prostate cancer-vs-normal, prostate cancer-vs-renal cancer, prostate cancer-vs-urothelial cancer.

In some embodiments of the present invention, in the apparatus, the processor is configured to execute the classification method according to any one of items of the present invention based on the instruction stored in the memory device.

In some embodiments of the present invention, in the apparatus, the urogenital system tumor is one or more selected from the group consisting of prostate cancer, urothelial cancer and renal cancer;

preferably, the renal cancer is clear renal cell carcinoma,

preferably, the urothelial cancer is upper urothelial cancer and/or bladder cancer,

preferably, the prostate cancer is prostate adenocarcinoma.

Another aspect of the present invention relates to a use of any one selected from the group consisting of the following items 1) to 3) in the manufacture of a medicament for detection, diagnosis, disease risk assessment or prognosis assessment of a human urogenital system tumor:

1) the biomarker combination according to any one of items of the present invention;

2) a cfDNA in a human urine, especially a cfDNA in a human urine supernatant;

preferably, the urine is a morning urine;

preferably, the cfDNA is cfDNA of 90 to 300 bp, or cfDNA of 100 to 300 bp; more preferably, the cfDNA is cfDNA of 90 to 150 bp, or cfDNA of 100 to 150 bp;

3) a DNA library, which is prepared by item 2); preferably, the DNA library is a whole genome library;

preferably, the urogenital system tumor is one or more selected from the group consisting of prostate cancer, urothelial cancer and renal cancer;

preferably, the renal cancer is clear renal cell carcinoma,

preferably, the urothelial cancer is upper urothelial cancer and/or bladder cancer,

preferably, the prostate cancer is prostate adenocarcinoma.

Another aspect of the present invention relates to any one selected from the group consisting of the following items 1) to 3), which is used for the detection, diagnosis, disease risk assessment or prognosis assessment of a human urogenital system tumor:

1) the biomarker combination according to any one of items of the present invention;

2) a cfDNA in a human urine, especially a cfDNA in a human urine supernatant;

Preferably, the urine is a morning urine;

Preferably, the cfDNA is cfDNA of 90 to 300 bp, or cfDNA of 100 to 300 bp; more preferably, the cfDNA is cfDNA of 90 to 150 bp, or cfDNA of 100 to 150 bp;

3) a DNA library, which is prepared by item 2); preferably, the DNA library is a whole genome library;

preferably, the urogenital system tumor is one or more selected from the group consisting of prostate cancer, urothelial cancer and renal cancer;

preferably, the renal cancer is clear renal cell carcinoma,

preferably, the urothelial cancer is upper urothelial cancer and/or bladder cancer,

preferably, the prostate cancer is prostate adenocarcinoma.

Another aspect of the present invention relates to a biomarker combination, which comprises m biomarkers, and m represents a positive integer greater than or equal to 50;

the biomarker is a DNA fragment, correspondingly having an initiate site of A±n1, and a termination site of B±n2 on the chromosome;

wherein, the n1 and n2 are independently non-negative integers less than or equal to 60,000;

wherein, the chromosome, A and B are any one group, any two groups, any three groups, any four groups, any five groups, any six groups (for example, the first 6 groups) or all 7 groups selected from the group consisting of the following Groups (1) to (7);

(1) Biomarkers for Renal Cancer Vs. Normal (the Smaller of the No. of the Biomarkers, the Higher of the Classification Effectiveness)

TABLE 1

| No. | Chromosome | A | B |
|---|---|---|---|
| 1 | chr14 | 105173382 | 105228468 |
| 2 | chr4 | 126141989 | 126199070 |
| 3 | chr2 | 38340335 | 38396819 |
| 4 | chr4 | 120896519 | 120952988 |
| 5 | chr1 | 225263465 | 225322410 |
| 6 | chr3 | 49627990 | 49683004 |
| 7 | chr12 | 55710185 | 55770826 |
| 8 | chr2 | 198023323 | 198078345 |
| 9 | chr8 | 104278540 | 104334789 |
| 10 | chr15 | 102366051 | 102531392 |
| 11 | chr5 | 56684537 | 56739554 |
| 12 | chr12 | 2875899 | 2930969 |
| 13 | chr5 | 8084151 | 8143261 |
| 14 | chr13 | 24239617 | 24294704 |
| 15 | chr14 | 63064067 | 63121825 |
| 16 | chr10 | 32966493 | 33022298 |
| 17 | chr18 | 34499871 | 34555093 |
| 18 | chr18 | 27538044 | 27593083 |
| 19 | chr19 | 52518298 | 52574358 |
| 20 | chr3 | 148084127 | 148140439 |
| 21 | chr11 | 23395282 | 23450515 |
| 22 | chr19 | 53868391 | 53924718 |
| 23 | chr7 | 36856760 | 36911789 |
| 24 | chr19 | 55851675 | 55906675 |
| 25 | chr12 | 130622755 | 130677832 |
| 26 | chr8 | 88140900 | 88196181 |
| 27 | chr8 | 98015299 | 98073611 |
| 28 | chr22 | 24279186 | 24375790 |
| 29 | chr10 | 58285076 | 58342675 |
| 30 | chr1 | 193398457 | 193455292 |
| 31 | chr11 | 44170591 | 44225937 |
| 32 | chr3 | 99497035 | 99552049 |
| 33 | chr18 | 70229325 | 70284364 |
| 34 | chr3 | 86800483 | 86855497 |
| 35 | chr7 | 85391699 | 85446714 |
| 36 | chr2 | 222217699 | 222274614 |
| 37 | chr12 | 51953090 | 52017679 |
| 38 | chr2 | 231506603 | 231561625 |
| 39 | chr7 | 54479671 | 54534725 |
| 40 | chr5 | 40826473 | 40882045 |
| 41 | chr3 | 61041867 | 61097030 |
| 42 | chr1 | 71530378 | 71587704 |
| 43 | chr19 | 30375804 | 30434948 |
| 44 | chr5 | 103365336 | 103426037 |
| 45 | chr16 | 72331875 | 72390386 |
| 46 | chr12 | 77381964 | 77436979 |
| 47 | chr19 | 35419205 | 35474205 |
| 48 | chr8 | 131286269 | 131341291 |
| 49 | chr21 | 30776557 | 30834320 |
| 50 | chr9 | 17638202 | 17695124 |

(2) Biomarkers for Urothelial Carcinoma Vs. Normal (the Smaller of the No. of the Biomarkers, the Higher of the Classification Effectiveness)

TABLE 2

| No. | Chromosome | A | B |
|---|---|---|---|
| 1 | chr1 | 165542998 | 165598528 |
| 2 | chr20 | 45298182 | 45353725 |
| 3 | chr7 | 110250206 | 110305749 |
| 4 | chr8 | 34086369 | 34141392 |
| 5 | chr11 | 3080528 | 3135556 |
| 6 | chr8 | 81773551 | 81828573 |
| 7 | chr7 | 20604578 | 20660880 |
| 8 | chr8 | 101664207 | 101719230 |
| 9 | chr8 | 127300805 | 127363897 |
| 10 | chr3 | 175419548 | 175474633 |
| 11 | chr7 | 17433047 | 17488061 |
| 12 | chr11 | 126763962 | 126818990 |
| 13 | chr8 | 81328435 | 81383788 |
| 14 | chr1 | 160347268 | 160402416 |
| 15 | chr3 | 150917292 | 150976246 |
| 16 | chr8 | 78266536 | 78321853 |
| 17 | chr2 | 127233784 | 127288805 |
| 18 | chr9 | 119009696 | 119064910 |
| 19 | chr7 | 88363140 | 88418154 |
| 20 | chr6 | 168087004 | 168142398 |
| 21 | chr8 | 101056393 | 101111465 |
| 22 | chr9 | 121669613 | 121725772 |
| 23 | chr8 | 32804682 | 32859711 |
| 24 | chr1 | 160016845 | 160071870 |
| 25 | chr8 | 52860841 | 52916007 |
| 26 | chr1 | 184863212 | 184918237 |
| 27 | chr8 | 103059578 | 103114914 |
| 28 | chr11 | 131771420 | 131826541 |
| 29 | chr11 | 132772276 | 132827397 |
| 30 | chr8 | 142309304 | 142365059 |
| 31 | chr11 | 20866407 | 20922555 |
| 32 | chr9 | 9389289 | 9445177 |
| 33 | chr8 | 86975952 | 87030974 |
| 34 | chr8 | 68297698 | 68353353 |
| 35 | chr9 | 122009782 | 122064791 |
| 36 | chr8 | 61387868 | 61442890 |
| 37 | chr8 | 82499446 | 82554469 |
| 38 | chr9 | 118116705 | 118171814 |
| 39 | chr8 | 117772819 | 117827841 |
| 40 | chr9 | 135838140 | 135893149 |
| 41 | chr14 | 101522031 | 101577065 |
| 42 | chr8 | 81105039 | 81160812 |
| 43 | chr3 | 161042779 | 161098402 |
| 44 | chr9 | 104364444 | 104420690 |
| 45 | chr8 | 61111592 | 61166615 |
| 46 | chr20 | 31048866 | 31103880 |
| 47 | chr15 | 26890253 | 26945265 |
| 48 | chr4 | 28406811 | 28462319 |
| 49 | chr5 | 35031116 | 35086691 |
| 50 | chr10 | 101035266 | 101090283 |

(3) Biomarkers for Prostate Cancer Vs. Normal (the Smaller of the No. of the Biomarkers, the Higher of the Classification Effectiveness)

TABLE 3

| No. | Chromosome | A | B |
|---|---|---|---|
| 1 | chr6 | 150259849 | 150319419 |
| 2 | chr11 | 50065867 | 50143253 |
| 3 | chr2 | 223609354 | 223664376 |
| 4 | chr3 | 178315458 | 178370471 |
| 5 | chr5 | 142022744 | 142077815 |
| 6 | chr3 | 72366362 | 72421541 |
| 7 | chr14 | 51571751 | 51628678 |
| 8 | chr10 | 69911981 | 69966998 |
| 9 | chr9 | 75793867 | 75850925 |
| 10 | chr16 | 34486643 | 34542808 |
| 11 | chr16 | 75960918 | 76016022 |
| 12 | chr1 | 213593324 | 213648410 |
| 13 | chr14 | 81176000 | 81231314 |
| 14 | chr14 | 48680148 | 48735914 |
| 15 | chr1 | 66328295 | 66385662 |
| 16 | chr2 | 236695859 | 236750881 |
| 17 | chr16 | 34310644 | 34370518 |
| 18 | chr13 | 70644019 | 70699054 |
| 19 | chr1 | 104971030 | 105026648 |
| 20 | chr19 | 20033425 | 20088912 |
| 21 | chr12 | 41633765 | 41689196 |
| 22 | chr1 | 111186072 | 111241148 |
| 23 | chr11 | 81515081 | 81570551 |
| 24 | chr6 | 164934635 | 164990438 |
| 25 | chr7 | 88753879 | 88809024 |
| 26 | chr2 | 204421512 | 204476533 |
| 27 | chr13 | 38205109 | 38260137 |
| 28 | chr19 | 57310235 | 57365579 |
| 29 | chr5 | 172615261 | 172670278 |
| 30 | chr13 | 100608580 | 100663608 |
| 31 | chr1 | 248513391 | 248569321 |
| 32 | chr5 | 78269787 | 78325922 |
| 33 | chr10 | 12753021 | 12808156 |
| 34 | chr7 | 101911102 | 101966116 |
| 35 | chr17 | 30274080 | 30334227 |
| 36 | chr12 | 87935928 | 87995848 |
| 37 | chr9 | 12175965 | 12231559 |
| 38 | chr5 | 97385699 | 97441111 |
| 39 | chr8 | 3970051 | 4025074 |
| 40 | chr7 | 20604578 | 20660880 |
| 41 | chr8 | 32416104 | 32471278 |
| 42 | chr7 | 12021765 | 12077292 |
| 43 | chr20 | 11563548 | 11624648 |
| 44 | chr7 | 51785230 | 51840244 |
| 45 | chr19 | 16615231 | 16670336 |
| 46 | chr10 | 67343243 | 67399416 |
| 47 | chr11 | 10953369 | 11008630 |
| 48 | chr2 | 22332272 | 22390528 |
| 49 | chr17 | 10390372 | 10446415 |
| 50 | chr4 | 976667 | 1032082 |

(4) Biomarkers for Renal Cancer Vs. Prostate Cancer (the Smaller of the No. of the Biomarkers, the Higher of the Classification Effectiveness)

TABLE 4

| No. | Chromosome | A | B |
|---|---|---|---|
| 1 | chr4 | 163059481 | 163114735 |
| 2 | chr4 | 6580383 | 6635407 |
| 3 | chr6 | 132270265 | 132325276 |
| 4 | chr2 | 82257259 | 82312280 |
| 5 | chr1 | 159394058 | 159452969 |
| 6 | chr9 | 105154079 | 105209849 |
| 7 | chr2 | 187699497 | 187754518 |
| 8 | chr4 | 126199070 | 126254087 |
| 9 | chr20 | 18854392 | 18909406 |
| 10 | chr7 | 15040427 | 15095480 |
| 11 | chr3 | 44690964 | 44747019 |
| 12 | chr11 | 57212694 | 57267722 |
| 13 | chr2 | 48829261 | 48885035 |
| 14 | chr12 | 133782920 | 133851895 |
| 15 | chr5 | 98900964 | 98963876 |
| 16 | chr11 | 86090264 | 86145292 |
| 17 | chr7 | 128477838 | 128533737 |
| 18 | chr2 | 32933311 | 32988604 |
| 19 | chr7 | 12693292 | 12748805 |
| 20 | chr4 | 95879059 | 95934075 |
| 21 | chr8 | 59989616 | 60044780 |
| 22 | chr12 | 32405135 | 32460143 |
| 23 | chr7 | 37972210 | 38027551 |
| 24 | chr11 | 128601685 | 128656714 |
| 25 | chr6 | 64185537 | 64240615 |
| 26 | chr7 | 107787926 | 107843035 |
| 27 | chr18 | 29036127 | 29091424 |
| 28 | chr16 | 47711531 | 47767836 |
| 29 | chr7 | 14590286 | 14645354 |
| 30 | chr11 | 55525982 | 55582014 |
| 31 | chr5 | 174061726 | 174116744 |
| 32 | chr14 | 44456533 | 44512749 |
| 33 | chr3 | 168694552 | 168750070 |
| 34 | chr4 | 114652704 | 114707721 |
| 35 | chr2 | 27431778 | 27486799 |
| 36 | chr4 | 107314339 | 107370716 |
| 37 | chr2 | 182718295 | 182773317 |
| 38 | chr10 | 19690582 | 19745774 |
| 39 | chr10 | 23594781 | 23649798 |
| 40 | chr3 | 3972580 | 4034015 |
| 41 | chr6 | 31323092 | 31379758 |
| 42 | chr8 | 128874896 | 128929933 |
| 43 | chr1 | 26256318 | 26311633 |
| 44 | chr5 | 161340570 | 161395587 |
| 45 | chr12 | 91346168 | 91401202 |
| 46 | chr19 | 2637431 | 2692582 |
| 47 | chr7 | 36856760 | 36911789 |
| 48 | chr9 | 27809024 | 27864032 |
| 49 | chr2 | 116615151 | 116670172 |
| 50 | chr9 | 112566383 | 112621994 |

(5) Biomarkers for Urothelial Cancer Vs. Renal Cancer (the Smaller of the No. of the Biomarkers, the Higher of the Classification Effectiveness)

TABLE 5

| No. | Chromosome | A | B |
|---|---|---|---|
| 1 | chr4 | 163059481 | 163114735 |
| 2 | chr4 | 6580383 | 6635407 |
| 3 | chr6 | 132270265 | 132325276 |
| 4 | chr2 | 82257259 | 82312280 |
| 5 | chr1 | 159394058 | 159452969 |
| 6 | chr9 | 105154079 | 105209849 |
| 7 | chr2 | 187699497 | 187754518 |
| 8 | chr4 | 126199070 | 126254087 |
| 9 | chr20 | 18854392 | 18909406 |
| 10 | chr7 | 15040427 | 15095480 |
| 11 | chr3 | 44690964 | 44747019 |
| 12 | chr11 | 57212694 | 57267722 |
| 13 | chr2 | 48829261 | 48885035 |
| 14 | chr12 | 133782920 | 133851895 |
| 15 | chr5 | 98900964 | 98963876 |
| 16 | chr11 | 86090264 | 86145292 |
| 17 | chr7 | 128477838 | 128533737 |
| 18 | chr2 | 32933311 | 32988604 |
| 19 | chr7 | 12693292 | 12748805 |
| 20 | chr4 | 95879059 | 95934075 |
| 21 | chr8 | 59989616 | 60044780 |
| 22 | chr12 | 32405135 | 32460143 |
| 23 | chr7 | 37972210 | 38027551 |
| 24 | chr11 | 128601685 | 128656714 |
| 25 | chr6 | 64185537 | 64240615 |
| 26 | chr7 | 107787926 | 107843035 |
| 27 | chr18 | 29036127 | 29091424 |
| 28 | chr16 | 47711531 | 47767836 |
| 29 | chr7 | 14590286 | 14645354 |
| 30 | chr11 | 55525982 | 55582014 |
| 31 | chr5 | 174061726 | 174116744 |
| 32 | chr14 | 44456533 | 44512749 |
| 33 | chr3 | 168694552 | 168750070 |
| 34 | chr4 | 114652704 | 114707721 |
| 35 | chr2 | 27431778 | 27486799 |
| 36 | chr4 | 107314339 | 107370716 |
| 37 | chr2 | 182718295 | 182773317 |
| 38 | chr10 | 19690582 | 19745774 |
| 39 | chr10 | 23594781 | 23649798 |
| 40 | chr3 | 3972580 | 4034015 |
| 41 | chr6 | 31323092 | 31379758 |
| 42 | chr8 | 128874896 | 128929933 |
| 43 | chr1 | 26256318 | 26311633 |
| 44 | chr5 | 161340570 | 161395587 |
| 45 | chr12 | 91346168 | 91401202 |
| 46 | chr19 | 2637431 | 2692582 |
| 47 | chr7 | 36856760 | 36911789 |
| 48 | chr9 | 27809024 | 27864032 |
| 49 | chr2 | 116615151 | 116670172 |
| 50 | chr9 | 112566383 | 112621994 |

(6) Biomarkers for Urothelial Cancer Vs. Prostate Cancer (the Smaller of the No. of the Biomarkers, the Higher of the Classification Effectiveness)

TABLE 6

| No. | Chromosome | A | B |
|---|---|---|---|
| 1 | chr3 | 88025277 | 88080310 |
| 2 | chr19 | 39394315 | 39449482 |
| 3 | chr20 | 31436554 | 31491568 |
| 4 | chr7 | 48432792 | 48487842 |
| 5 | chr8 | 87141019 | 87196120 |
| 6 | chr4 | 13859414 | 13914431 |
| 7 | chr1 | 160292243 | 160347268 |
| 8 | chr8 | 112245103 | 112300126 |
| 9 | chr8 | 11530043 | 11585066 |
| 10 | chr8 | 13932292 | 13987366 |
| 11 | chr3 | 152913886 | 152973883 |
| 12 | chr9 | 109516082 | 109571205 |
| 13 | chr11 | 8343925 | 8398954 |
| 14 | chr3 | 122030664 | 122085678 |
| 15 | chr5 | 87727661 | 87782722 |
| 16 | chr5 | 60881889 | 60936907 |
| 17 | chr14 | 40518423 | 40573582 |
| 18 | chr8 | 94667609 | 94724236 |
| 19 | chr8 | 101719230 | 101774274 |
| 20 | chr5 | 113527635 | 113584160 |
| 21 | chr3 | 103853900 | 103909150 |
| 22 | chr8 | 62393903 | 62449668 |
| 23 | chr8 | 124248002 | 124303024 |
| 24 | chr17 | 74131207 | 74186417 |
| 25 | chr14 | 52519339 | 52574927 |
| 26 | chr3 | 144795549 | 144851338 |
| 27 | chr3 | 84803116 | 84858323 |
| 28 | chr8 | 50523567 | 50578589 |
| 29 | chr8 | 88545977 | 88603606 |
| 30 | chr1 | 42119088 | 42174113 |
| 31 | chr20 | 43860121 | 43915135 |
| 32 | chr9 | 121061199 | 121116207 |
| 33 | chr9 | 118676908 | 118734641 |
| 34 | chr11 | 13163841 | 13219126 |
| 35 | chr11 | 57212694 | 57267722 |
| 36 | chr8 | 131892873 | 131948409 |
| 37 | chr11 | 16410024 | 16465871 |
| 38 | chr8 | 109405759 | 109460782 |
| 39 | chr5 | 158002797 | 158058189 |
| 40 | chr11 | 1579888 | 1635511 |
| 41 | chr8 | 51749113 | 51804136 |
| 42 | chr9 | 118562723 | 118621899 |
| 43 | chr17 | 29154317 | 29209332 |
| 44 | chr6 | 73471411 | 73528437 |
| 45 | chr3 | 87522168 | 87578480 |
| 46 | chr1 | 231915581 | 231971963 |
| 47 | chr8 | 117772819 | 117827841 |
| 48 | chr1 | 241691293 | 241746318 |
| 49 | chr9 | 92506773 | 92712072 |
| 50 | chr4 | 19120611 | 19176371 |

(7) Biomarkers for Normal Vs. Prostate Cancer (Considering Gender Differences, Only the Male are Included in the Normal Population; the Smaller of the No. of the Biomarkers, the Higher of the Classification Effectiveness)

TABLE 7

| No. | Chromosome | A | B |
|---|---|---|---|
| 1 | chr11 | 40374531 | 40429896 |
| 2 | chr12 | 61310253 | 61365625 |
| 3 | chr19 | 56809188 | 56866674 |
| 4 | chr2 | 145644444 | 145702420 |
| 5 | chr6 | 98011442 | 98066653 |
| 6 | chr7 | 88753879 | 88809024 |
| 7 | chr9 | 98761758 | 98817567 |
| 8 | chrY | 4474368 | 4588559 |
| 9 | chrY | 18884928 | 18940043 |
| 10 | chrY | 5632826 | 5746826 |
| 11 | chrY | 24371813 | 24427746 |
| 12 | chrY | 5948790 | 6035624 |
| 13 | chrY | 19228861 | 19283946 |
| 14 | chrY | 21484883 | 21542276 |
| 15 | chrY | 5746826 | 5851679 |
| 16 | chrY | 28707448 | 28764196 |
| 17 | chrY | 6599942 | 6664881 |
| 18 | chrY | 23799512 | 23860617 |
| 19 | chrY | 3427018 | 3545705 |
| 20 | chrY | 13573548 | 13635016 |
| 21 | chrY | 18387555 | 18551943 |
| 22 | chrY | 16529414 | 16585431 |
| 23 | chrY | 19111726 | 19166891 |
| 24 | chrY | 9020782 | 9081054 |
| 25 | chrY | 19451088 | 19508211 |
| 26 | chrY | 6720180 | 6778075 |
| 27 | chrY | 6349316 | 6458079 |
| 28 | chrY | 4163770 | 4261597 |
| 29 | chrY | 28648165 | 28707448 |
| 30 | chrY | 8741265 | 8796960 |
| 31 | chrY | 19283946 | 19339589 |
| 32 | chrY | 3970433 | 4073487 |
| 33 | chrY | 7346142 | 7402799 |
| 34 | chrY | 15149848 | 15205024 |
| 35 | chrY | 18774055 | 18829409 |
| 36 | chrY | 7290613 | 7346142 |
| 37 | chrY | 23743018 | 23799512 |
| 38 | chrY | 4700163 | 4811039 |
| 39 | chrY | 16473510 | 16529414 |
| 40 | chrY | 21654324 | 21709511 |
| 41 | chrY | 14418460 | 14477812 |
| 42 | chrY | 5851679 | 5948790 |
| 43 | chrY | 8685630 | 8741265 |
| 44 | chrY | 14650141 | 14705375 |
| 45 | chrY | 15605187 | 15663531 |
| 46 | chrY | 4073487 | 4163770 |
| 47 | chrY | 9399760 | 9457656 |
| 48 | chrY | 4366038 | 4474368 |
| 49 | chrY | 4937971 | 5066009 |
| 50 | chrY | 19564127 | 21039220 |

In some embodiments of the present invention, in the biomarker combination, m is 50 to 300 or greater than 300, such as 50 to 100, 100 to 150, 150 to 200, 200 to 250, 250 to 300, 50, 100, 150, 200, 250, or 300.

In one or more embodiments of the present invention, in the biomarker combination, n1 and n2 are independently 5,000, 4,000, 3,000, 2,000, 1500, 1,000, 500, 300, 200, 150, 100, 90, 80, 70, 60, 50, 40, 30, 20, 10, 5 or 0.

In one or more embodiments of the present invention, in the biomarker combination, the biomarker is a fragment of cfDNA; preferably, the cfDNA is derived from a human urine, especially a human urine supernatant.

In one or more embodiments of the present invention, in the biomarker combination:

the chromosome, A and B are shown in any 1 group, any 2 groups, any 3 groups, any 4 groups, any 5 groups, any 6 groups, or all 7 groups selected from the group consisting of the Groups (1) to (7).

Some terms involved in the present invention are explained as follows.

The term “bin” (interval/region) refers to a general description in the field of genomics that artificially defines or divides a genome according to a certain length. For example, when about 3 billion base pairs of human genome are equally divided into 3,000 bin pairs, each bin has a size of about 1 million base pairs.

The term “cfNA” is the abbreviation of cell free nucleic acid, which refers to a free nucleic acid in plasma, which is an extracellular nucleic acid fragment in the peripheral circulation.

The term “cfDNA” is the abbreviation of cell free DNA, which refers to a free DNA in plasma, which is an extracellular DNA fragment in the peripheral circulation.

The term “coverage” refers to a proportion of a region of genome that has been detected at least once in the entire genome. Coverage is a term that measures the coverage degree that the genome is covered by data. Due to the existence of complex structures such as high GC and repetitive sequences in the genome, the sequence obtained by final splicing and assembling in the sequencing often cannot cover the entire genome, and the region that is not obtained is called Gap. For example, if a bacterial genome is sequenced to have a coverage of 98%, then 2% of the sequence region is not obtained through the sequencing.

The term “sequencing depth” refers to a ratio of the total number of bases (bp) obtained by sequencing to the size of genome (Genome), or can be understood as the average number of times that each base in the genome is sequenced. For example, if a gene is 2M in size and the total amount of data obtained is 20M, then the sequencing depth is 20M/2M=10×.

The term “read” or “reads” refers to reads, that is, the measured sequence.

The term “pair-end reads” refers to paired reads.

The term “copy number variations (CNVs)” refers to the deletion or duplication of larger DNA fragments, i.e., the common increase or decrease in the copy number of DNA fragments ranging from hundreds bp to millions bp. CNVs are caused by genome rearrangement and are one of the important pathogenic factors of tumors.

The term “theoretical simulation copy number” refers to the copy number calculated by a software and/or method, in which the division of the genome is divided into several regions with equal or unequal lengths, but through data simulation, the theoretical copy number contained in each region is the same.

The beneficial effects of the present invention

(1) Trace detection reduces the cost of sequencing, and the detection is achieved under a lower and shallower coverage. The content of cfDNA released by early tumor cells is generally less than one percent or even one ten thousandth. Therefore, it is very challenging and requires a very deep sequencing depth for the current DNA detection technology to detect variations at levels of SNV (single nucleic acid variation) and INDEL (insertion/deletion) in ctDNA. However, the present inventors use cfDNA whole-genome sequencing technology to detect the copy number variation, which is theoretically and technically feasible. The sample sequencing depth used by the present inventors is only 1× to 5×, and a highly sensitive and specific diagnosis is achieved.

(2) Highly accurate diagnosis of single urinary system tumor is achieved.

(3) Tissue specific diagnosis. The problem of what tumor is diagnosed under unknown circumstances is solved. Based on the biomarker groups selected by the established classification system, the present inventors can determine at one time with high accuracy that the sample comes from which tumor in the urinary system.

(4) Truly non-invasive. Urine collection is simple and non-invasive, and cause no pain in patients, which is conducive to sample collection, diagnosis, long-term and regular prognostic monitoring.

### Specific Models for Carrying Out the Invention

The embodiments of the present invention will be described in detail below in conjunction with examples, but those skilled in the art will understand that the following examples are only used to illustrate the present invention and should not be regarded as limiting the scope of the present invention. If specific conditions were not indicated in the examples, they would be carried out in accordance with the conventional conditions or the conditions recommended by the manufacturer. The reagents or instruments used without the manufacturer's indication were all conventional products that were purchased commercially.

### Example 1

Preparation of cfDNA Sample

### 1. Target Group

95 healthy people;

172 patients, comprising: 58 patients with clear renal cell carcinoma (ccRCC), 69 patients with urothelial carcinoma and 45 patients with prostate cancer. All were diagnosed by tissue biopsy of surgical samples.

There were a total of 267 cases of healthy persons and patients.

### 2. Experimental Method

(1) Morning urine of the above-mentioned healthy persons and preoperative morning urine of tumor patients were collected. The urine of each case was collected in a 50 ml tube with about 20 to 50 ml. After collection, urine was placed in an ice box, and extracted within half hour to avoid degradation of cfDNA.

(2) The collected morning urine were centrifuged at 3500 rpm for 15 minutes, and then their supernatants were remained respectively.

(3) The cfDNA was extracted using zymo Quick-DNA™ Urine Kit. The concentrations were measured with Qubit4 Fluorometer, and they were stored at −80° C.

267 cfDNA samples were prepared.

### Example 2

### Construction of the Whole Genome Library

### 1. Experimental Samples, Reagents and Instruments

The 267 cfDNA samples obtained in Example 1 above.

Extraction kit for free urine DNA: ZYMO Quick to DNA Urine Kit (ZYMO, Cat #: D3061).

Magnetic beads: AMPure XP beads (Beckman Coulter, Cat #: A63880).

Regular centrifuge.

### 2. Experimental Method

(1) cfDNA of 100 bp to 300 bp was screened by magnetic beads (the range of size of the DNA fragments binded by the magnetic beads were controlled by the ratio of the volume of the magnetic beads to the volume of the cfDNA sample). The specific operations were as follows:

To extract urine cfDNA, 0.6 times of magnetic beads was added, the magnetic beads were discarded after binding for 5 minutes, the supernatant was retained, then 0.3 times of magnetic beads were added to the supernatant, the supernatant was discarded after binding for 5 minutes, and the magnetic beads were retained (notation: the purpose of adding 0.6 times the volume of magnetic beads was to bind large DNA fragments that were then discarded, and the addition of 0.3 times the volume of magnetic beads to the supernatant was to bind small fragments as target DNA fragments, thus the small DNA fragments were recovered), wash twice with 80% ethanol, and finally the DNA was dissolved with water.

(2) End-repair and adding A. The specific operations were performed by referring to the instructions of kits, NEBNext End Repair Module: catalog number E6050S; NEBNext dA-Tailing Module, catalog number E6053S.

(3) Adding PE adaptor. The specific operations were performed by referring to the operating instructions of kit, T4 DNA Ligase, catalog number M0202L.

(4) A adaptor-specific primer was used for PCR amplification.

(5) The PCR product obtained above was purified with magnetic beads to obtain the DNA library, i.e., the whole genome library of each sample from 267 cases.

In addition, Agilent 2100 Bioanalyser was used to conduct quality detection of the 267 libraries, and there was no adaptor contamination after the library was constructed.

### Example 3

### HiSeq X10 Sequencing

### 1. Reagents and Instruments

Samples to be tested: the libraries of the 267 cases prepared in Example 2 above.

### 2. Experimental Method

Whole-genome sequencing was performed. The sequencing was commissioned to Novagene Sequencing Company.

### 3. Experimental Results

50 bp pair-end reads from 267 libraries were obtained. The sequencing depth of each sample was approximately 1× to 5×. These were used for the following tumor marker analysis.

### Example 4

### Screening, Analysis and Application of Tumor Markers

### 1. Experimental Method

### (1) Calculation of Ratio A/B

According to the Varbin algorithm (Genome-wide copy number analysis of single cells. Nature protocols 7, 1024 to 1041, doi:10.1038/nprot.2012.039 (2012)), the genome of each sample was first divided into 50,000 bins, and then the number of reads and GC content in each bin were calculated in combination with the sequencing results of above Example 3, and the total number of reads and GC content obtained by sequencing each library sample were normalized, so as to obtain the original number of reads and the actual number of reads (A) corrected by GC content in each bin of each sample, in which the correction method was locally weighted scatterplot smoothing method (LOWESS smoothing); and the ratio A/B of the number of reads in each bin to the theoretical number of reads in the bin was further obtained:

A represented the actual number of reads in a bin after GC content correction;

B represented the theoretical number of reads in the bin, which was obtained by dividing the total number of reads measured in the sample by the total number of bins (50,000). Therefore, for a sample, the theoretical number of reads in each of its bins was equal.

The ratio A/B of greater than 1 indicated that this region was likely to have an increased copy number, equal to 1 indicated that this region had not changed, and less than 1 indicated that this region was likely to have a decreased copy number.

In the end, each sample got 50,000 ratios, and these 50,000 ratios (also called features) were used for the subsequent screening of markers.

### (2) Screening of Markers

For the 4 groups of object samples (healthy person samples, clear renal cell carcinoma patient samples, urothelial cancer patient samples, and prostate cancer patient samples), the object samples of each group were randomly divided into a training set (about 70%) and a test set (about 30%), so that 4 training sets and the corresponding 4 test sets were obtained respectively, and their respective numbers were shown in Table 8 below.

TABLE 8

| | Number of | Number of | Number of |
|---|---|---|---|
| Object group | each group | training set | test set |
| Healthy person samples | 95 | 67 | 28 |
| Clear renal cell carcinoma | 58 | 41 | 17 |
| patient samples | | | |
| Urothelial cancer patient | 69 | 48 | 21 |
| samples | | | |
| Prostate cancer patient samples | 45 | 32 | 13 |

First, pairwise comparison was made among the 4 training sets. Specifically, each bin was subjected to pairwise comparison between different groups, and the comparison was performed successively until all 50,000 bins were checked. That was, t test was performed on the ratios A/B corresponding to 50,000 bins, and when a ratio A/B with significant difference (p<0.05) was screened out by the t test, the marker (bin) corresponding to the ratio A/B was found. For example, a bin was taken, the ratio A/B corresponding to the bin of the normal person group was compared to that of the renal cancer group, and the bin was retained when the statistical test showed significant difference, otherwise, it was discarded; and such calculation was performed on the 50,000 bins. In this way, a total of 6 pairwise combinations and 6 groups of markers with significant differences were obtained.

Then these 6 groups of markers were further screened by a specific method comprising: performing binary classification model training by inputting the ratios A/B corresponding to the 6 groups of markers into the random forest classifier, performing sorting on the basis of feature importance (that was, the operation results of random forest algorithm) (the more important the marker was for the classification, the higher its sort order was), selecting the top markers such as top500, top300, top100, top50, top10 to perform the random forest model training again, evaluating the prediction accuracy rates of the training set and the test set under different marker sets, selecting the markers with high accuracy rates as the final marker set (when the accuracy rates were basically the same, the present inventors tended to choose a smaller number of marker combinations), and thus obtaining a total of 6 groups of markers by the 6 random forest binary classifiers, each group containing 50 markers as shown in the previous Table 1 to Table 6.

The data corresponding to the 6 groups of biomarkers (markers) in Table 1 to Table 6 (the ratios A/B of the 6 maker groups) were separately extracted, and used for training by the random forest algorithm, so as to finally obtain 6 binary classification models.

### (3) Construction of Integrated Classification System (GUdetector)

The present inventors combined these 6 binary classification models to perform multi-category classification by voting, and the specific method was as follows:

the present inventors designed 4 decision-making units, and each decision-making unit contained 3 random forest binary classifiers:

I. ‘normal decision-making unit’: normal-vs-renal cancer, normal-vs-urothelial cancer, normal-vs-prostate cancer;

II. ‘renal cancer decision-making unit’: renal cancer-vs-normal, renal cancer-vs-urothelial cancer, renal cancer-vs-prostate cancer;

III. ‘urothelial cancer decision-making unit’: urothelial cancer-vs-normal, urothelial cancer-vs-renal cancer, urothelial cancer-vs-prostate cancer;

IV. ‘prostate cancer decision-making unit’: prostate cancer-vs-normal, prostate cancer-vs-renal cancer, prostate cancer-vs-urothelial cancer.

Then the present inventors performed voting for each decision-making unit, that was, the ratios A/B of the 6 groups of markers corresponding to a sample were separately input into the respective classifiers of the above 4 decision-making units to perform prediction classification, for example, ‘normal decision-making unit’ got votes N₁ in prediction of the normal group, ‘renal cancer decision-making unit’ got votes N₂ in prediction of the renal cancer group, ‘prostate cancer decision-making unit’ got votes N₃ in prediction of the prostate cancer group, ‘urothelial cancer decision-making unit’ got votes N₄ in prediction of the urothelial cancer group; finally, the category corresponding to the decision-making unit with the highest number of votes is the finally predicted category, and if there were groups with the same number of votes, the category with the highest prediction probability in the groups with the same number of votes was the finally predicted category.

At the same time, the 6 groups of markers were subjected to the verification of reliability in the public TCGA database. The TCGA contained the copy number data of various tumor tissues (data of primary tumor tissues and normal tissues), the corresponding four sets of data were downloaded, then the values corresponding to the 6 groups of markers were calculated (the segment values provided by TCGA were used to measure the change in copy number) and input into the random forest model for training and prediction, and the accuracy was evaluated.

### 2. Analysis Results of Markers:

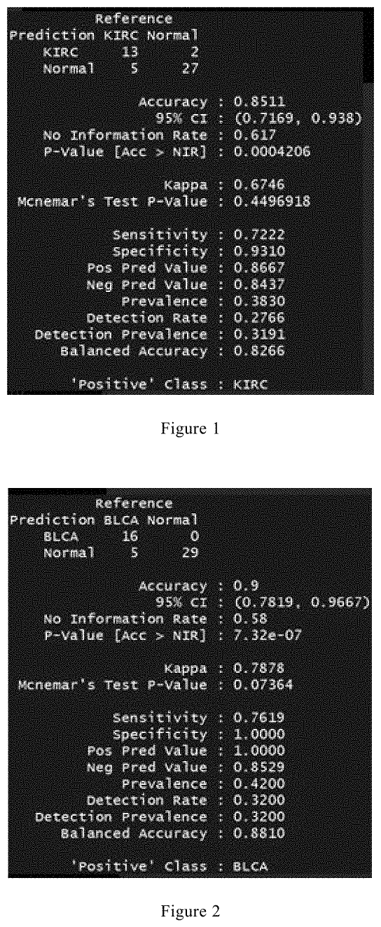

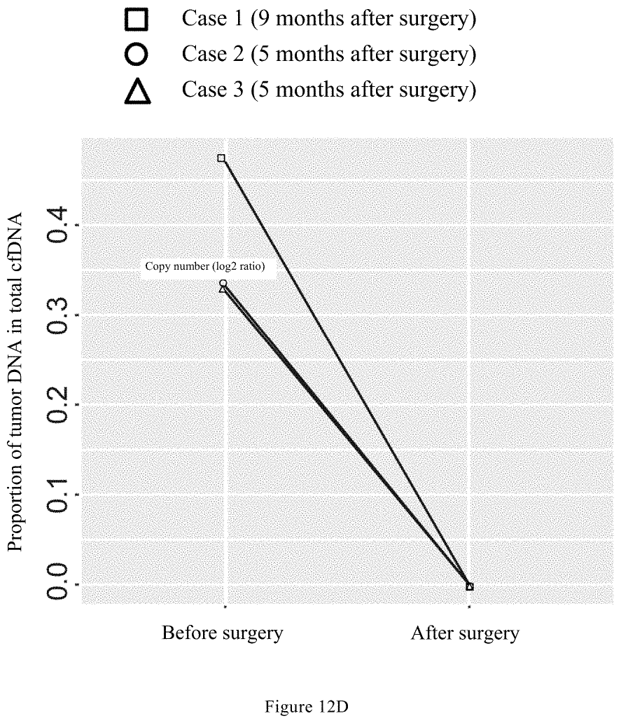

As shown in FIG. 1 to FIG. 12 (FIGS. 12A to 12D), in which KIRC represented renal cancer, UC represented urothelial cancer, PRAD represented prostate cancer, and Normal represented healthy person. The prediction results were all derived from the 30% test set. Generally, the training set was used to select markers and train the classification model, and the test set was used to evaluate the prediction accuracy.

The analysis results were the calculation results of the final 6 groups of markers that were selected, which were obtained by the classification performance evaluated by the random forest binary classifier and calculated by the function in the R language.

### 1) As Shown in FIG. 1.

Renal cancer vs. normal: sensitivity was 72.2%, specificity was 93.1%.

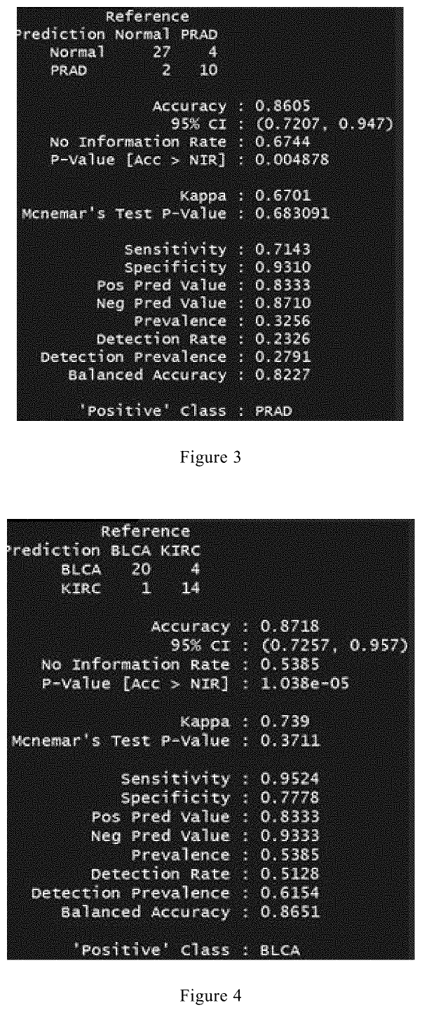

### 2) As Shown in FIG. 2.

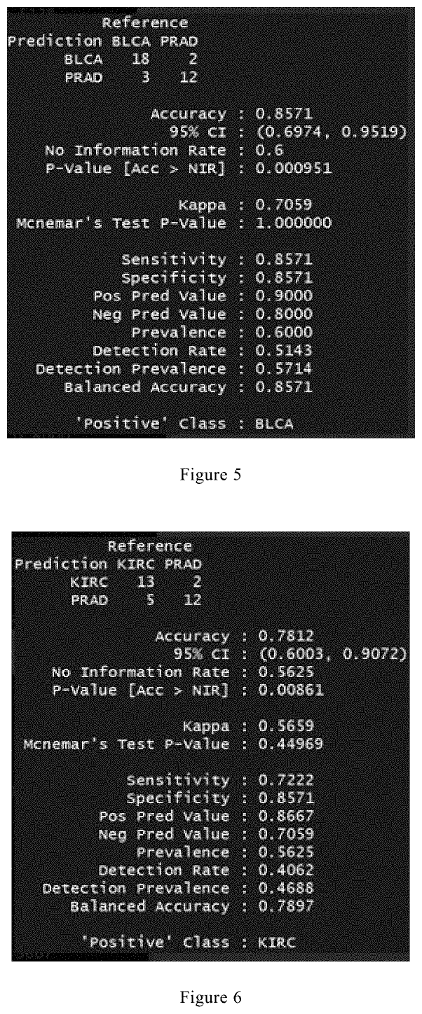

Urothelial carcinoma vs. normal: sensitivity was 76.2%, specificity was 100%. 3) As shown in FIG. 3.

Prostate cancer vs. normal: sensitivity was 71.4%, specificity was 93.1%.

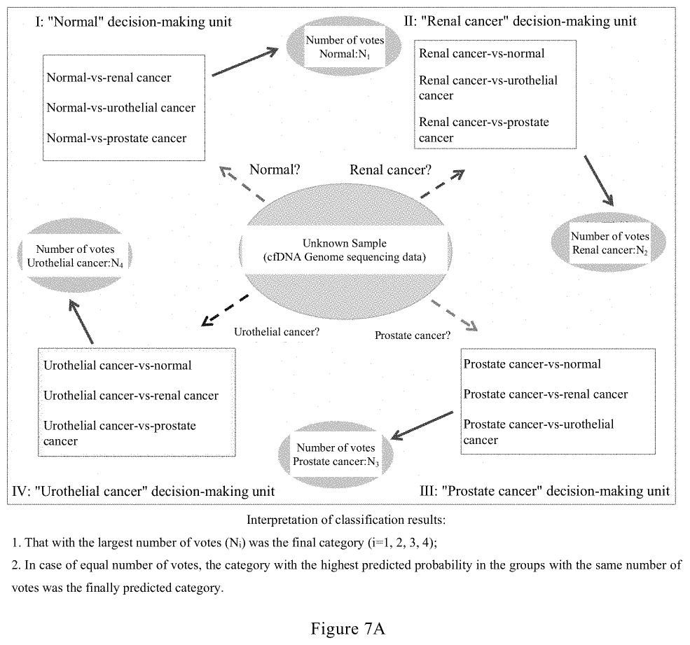

### 4) As Shown in FIG. 4.

Renal cancer vs. prostate cancer: sensitivity was 72.2%, specificity was 85.7%.

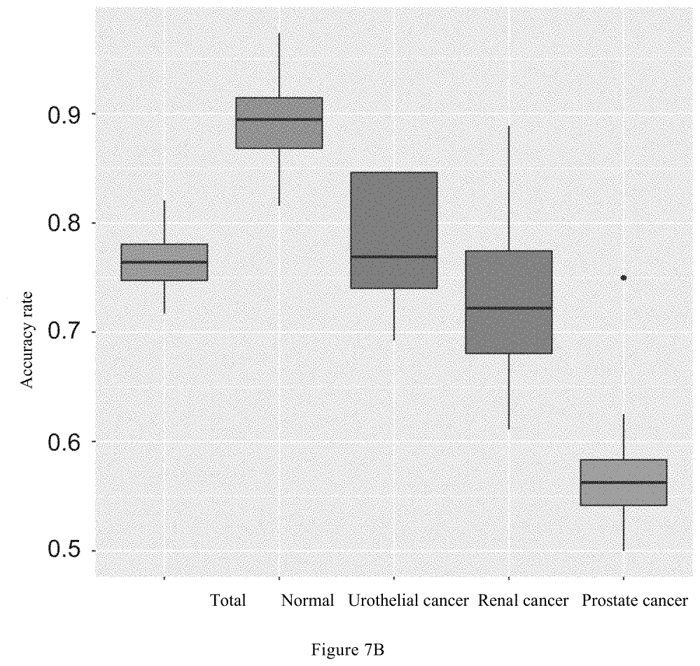

### 5) As Shown in FIG. 5.

Urothelial cancer vs. renal cancer: sensitivity was 95.2%, specificity was 77.8%.

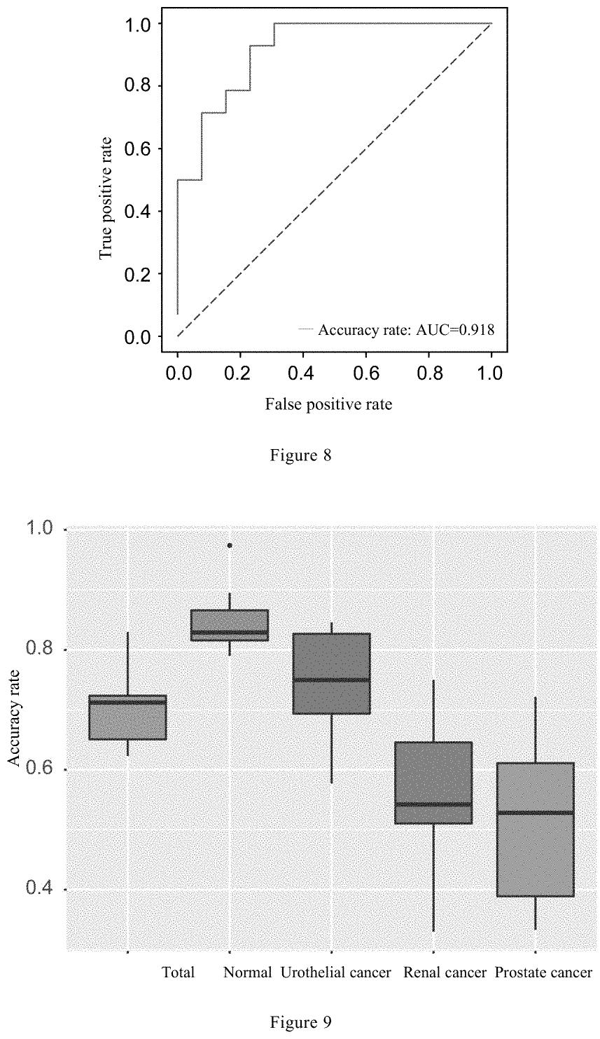

### 6) As Shown in FIG. 6.

Urothelial carcinoma vs prostate cancer: sensitivity was 85.7%, specificity was 85.7%.

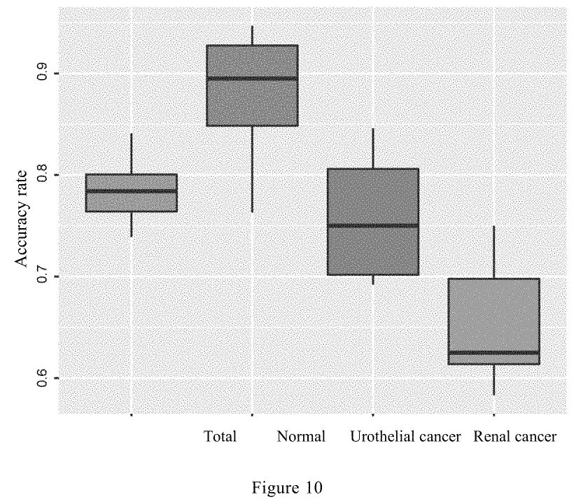

### 7) As Shown in FIG. 7A and FIG. 7B.

The experimental methods and samples in Examples 1 to 3 were referred to. Integrated classification system (GUdetector) was used for the simultaneous classification of the 4 groups.

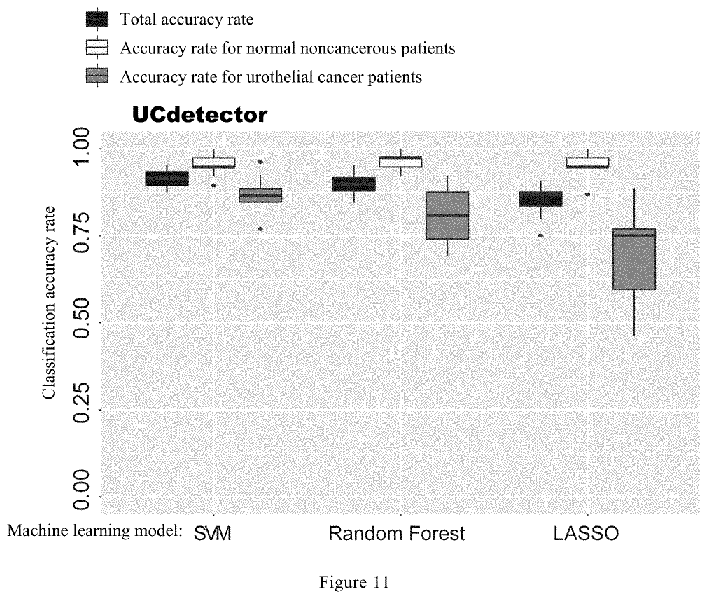

### 8) As Shown in FIG. 8.

Diagnosis model of prostate cancer for male samples. The experimental methods and samples in Examples 1 to 3 were referred to, and the copy number data of 43 male patients in the non-tumor population and 45 prostate cancer patients were used to construct the classification model.

Prostate cancer vs. normal: accuracy rate AUC=0.967.

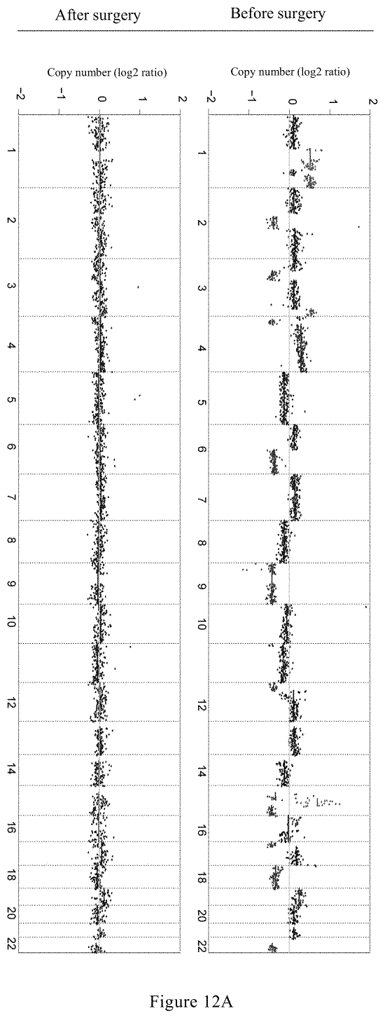

### 9) As Shown in FIG. 9.

Considering the gender factor, the markers on all sex chromosomes were removed, the experimental methods and samples in Examples 1 to 3 were referred to, and the SVM model was used for the simultaneous classification of the 4 groups.

The prediction accuracy rate for each category was: 89.7% for the normal group, 76.2% for the urothelial cancer group, 64.3% for the prostate cancer group, 44.4% for the renal cancer group, and the overall accuracy rate was 72.0%.

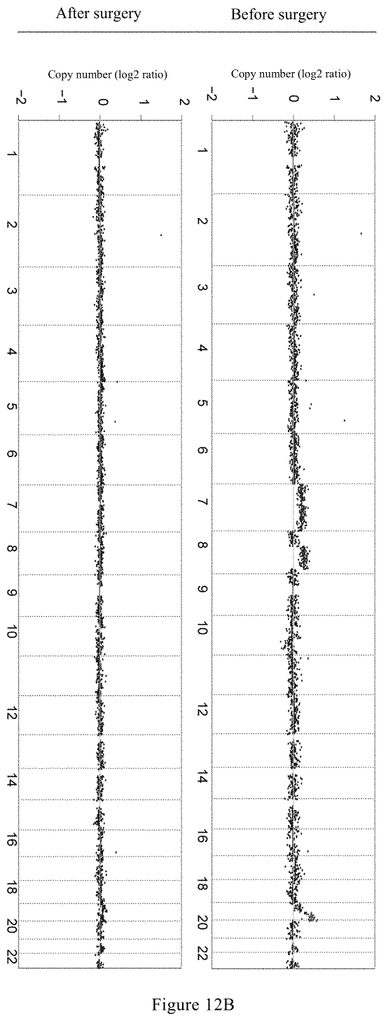

### 10) As Shown in FIG. 10.

The experimental methods and samples in Examples 1 to 3 were referred to, the SVM model was used to perform the simultaneous classification of the 3 groups, the results showed that the prediction accuracy rate for each category was: 88.5% for the normal group, 76.1% for the urothelial cancer group, 64.8% for the renal cancer group, and the overall accuracy rate was 78.4%.

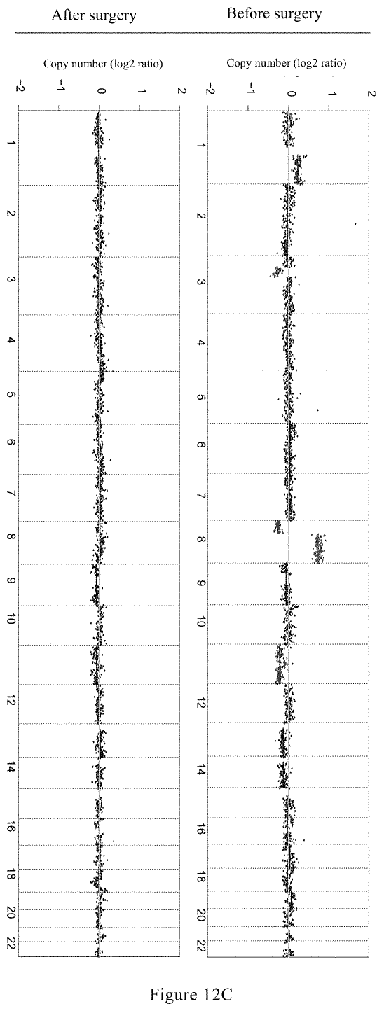

### 11) As Shown in FIG. 11.

The experimental methods and samples in Examples 1 to 3 were referred to, only 90 non-tumor individuals and 65 patients with urothelial cancer were used, and the SVM model was used to perform the diagnosis of urothelial cancer and compared with the LASSO and random forest methods. For the SVM, the prediction accuracy rate was 94.7% for the normal group, 86.5% for the urothelial cancer group, and the overall accuracy rate was 91.4%. For the LASSO, the prediction accuracy rate was 94.7% for the normal group, 75.0% for urothelial cancer group, and the overall accuracy rate was 86.72%. For random forest method, the prediction accuracy rate was 97.4% for the normal group, 80.8% for the urothelial cancer group, and the overall accuracy rate was 89.8%.

### 12) As Shown in FIG. 12A to 12D.

The experimental methods and samples in Examples 1 to 3 were referred to, the dynamic monitoring of therapeutic effect was exemplarily performed in 3 cases of urothelial cancer patients, before and after the operation of the 3 patients, the copy number of cfDNA and the proportion of tumor DNA in the total cfDNA were obtained by the ichorCNA algorithm. It could be seen that in all three patients, the copy number changes and tumor DNA content were detected before the operation, but they were not detected after the operation. This was consistent with the other tests of the patients. There was no recurrence in the three patients. The above results support that the present invention could also be used for non-invasive prognosis monitoring.

It was also noted that: Specificity and sensitivity are indicators to evaluate the efficiency of marker classification. Sensitivity refers to the ability to pick out cancer patients, and specificity refers to the ability to pick out normal people. For example, if there are 1,000 tumor patients and 1,000 normal persons, the present inventors could pick out 722 patients from the tumor group and 931 persons from the normal group by the classifier with sensitivity of 72.2% and specificity of 93.1%.

The sensitivity and specificity between two cancers refers to the ability to separate two tumors. Although these two concepts are used to evaluate negative and positive, or normal and abnormal, the present inventors herein also used them to evaluate two kinds of tumors, and the present inventors defined positive class, which was displayed as ‘positive’ class at the bottom of result.

In addition to the sensitivity value and specificity value, accuracy refers to the overall accuracy rate. The confusion matrix at the top of each result indicates the number correctly classified into a group and the number misclassified into another group.

Confusion matrix (Confusion matrix), Reference refers to the original category, Prediction refers to the predicted category, for example, the UC group, 16 UCs were predicted to be UC (predicted correctly), 2 UCs were predicted to be Normal, and 3 UC were predicted to be PRAD, none of them were predicted to be KIRC, and so forth;

the overall accuracy rate was 0.7195;

the prediction accuracy rate of each category was the corresponding Sensitivity below, and the specificity was not considered herein, because these two concepts were concepts of the classification for two categories, and the present classification was for 4 categories in which only the overall accuracy rate and the sensitivity of each category should be taken into account.

### 3. Discussion of Results:

The present inventors first established a urine-based cfDNA copy number classification system, which could predict the different tissue sources of unknown urogenital system tumors at one time through the screened biomarker groups, and had high sensitivity and specificity. In addition, considering gender differences, only men had the need to assess the risk of prostate cancer. Therefore, the present inventors also retrained prostate cancer classification markers for men. In addition, excluding gender factors, three classification models of normal, renal cancer and urothelial cancer were trained. Since the ensemble classification voting method could not be used for the classification of 3 categories, the present inventors compared machine learning classification methods such as SVM, LASSO and random forest, and found that the SVM model was significantly better than the other two machine algorithm models (LASSO and random forest).

### Example 5

### Diagnosis Example

For a random unknown subject in the outpatient clinic (who could be a healthy person, or a patient with urogenital system tumor), the following method was referred to:

1. collecting morning urine, and extracting cfDNA;

2. screening DNA fragments of 100 bp to 300 bp with magnetic beads,

3. construction of whole genome library;

4. performing the whole-genome sequencing on the library to obtain sequencing data;

5. dividing the genome of the sample into 50,000 bins; normalizing the sequencing data, and using the varbin algorithm to calculate the reads ratios corresponding to the 50,000 bins;

6. extracting the ratios corresponding to the 300 markers shown in Table 1 to Table 6, and inputting them into the above integrated classification system (GUdetector) for prediction.

The specific operations of the above steps 1 to 4 were referred to Examples 1 to 4 respectively.

### Example 6

### Screening of Diagnostic Markers for Prostate Cancer in Consideration of Gender Differences

Prostate cancer is a male-specific tumor. Therefore, if gender factors were not taken into account, since healthy people comprised males and females, the number of copies of sex chromosomes would overestimate the diagnostic accuracy of the classifier. Therefore, when the inventors of the present invention diagnosed whether an unknown male object had prostate cancer, men of healthy population were used for re-screening of markers (healthy men vs. prostate cancer patients, Table 7). For a male subject in the outpatient clinic, the following method was referred to:

1. collecting a morning urine and extracting cfDNA;

2. screening DNA fragments of 100 bp to 300 bp with magnetic beads,

3. construction of whole genome library;

4. performing the whole-genome sequencing on the library to obtain sequencing data;

5. dividing the genome of the sample into 50,000 bins; normalizing the sequencing data, and using the varbin algorithm to calculate the reads ratios corresponding to the 50,000 bins;

6. extracting the ratios corresponding to the 50 markers shown in Table 7, and using a machine learning algorithm such as SVM to predict whether the unknown sample was a prostate cancer patient.

The specific operations of the above steps 1 to 4 were referred to Examples 1 to 4 respectively.

### Example 7

### Screening of Markers for Diagnosis and Classification of Normal Person, Renal Cell Cancer Patient and Urothelial Cancer Patient

For a random unknown subject in the outpatient clinic (who could be a healthy person, or a patient with renal cancer and urothelial cancer), the following method was referred to:

1. collecting a morning urine and extracting cfDNA;

2. screening DNA fragments of 100 bp to 300 bp with magnetic beads,

3. construction of whole genome library;

4. perform the whole-genome sequencing on the library to obtain sequencing data;

5. dividing the genome of the sample into 50,000 bins; normalizing the sequencing data, and using the varbin algorithm to calculate the reads ratios corresponding to the 50,000 bins;

6. extracting the ratios corresponding to the 150 markers shown in Tables 1, 2 and 5, and using a machine learning algorithm such as SVM to predict whether the unknown sample was normal person, renal cancer patient, or urothelial cancer patient.

The specific operations of the above steps 1 to 4 were referred to Examples 1 to 4 respectively.

### Example 8

### Example of Dynamic Monitoring of Therapeutic Efficacy of Urothelial Cancer

The copy number analysis of cfDNA could be obtained by other algorithms, such as the ichorCNA algorithm. In this method, the genomic region was divided into uniform regions with a length of 1,000,000 bp, and then the copy number variation and the proportion of tumor-derived DNA were calculated. For a patient who was checked before surgery and rechecked after treatment in the outpatient clinic, the following method was referred to:

1. collecting a morning urine before surgery and a morning urine during regular review, and extracting cfDNA;

2. screening DNA fragments of 100 bp to 300 bp with magnetic beads,

3. construction of whole genome library;

4. performing the whole-genome sequencing on the library to obtain sequencing data;

5. using the ichorCNA method to obtain the copy number variation atlases of cfDNA in the urine of the cancer patient before surgery and in the urine during regular review, and estimating tumor DNA contents;

6. evaluating the treatment efficacy and recurrence of the patient according to the comparison of the above atlases and tumor DNA contents.

### Comparative Example 1

### Using LASSO Algorithm Model

### 1. Experimental Method

The method in the reference, Circulating tumour DNA methylation markers for diagnosis and prognosis of hepatocellular carcinoma, was used.

The input data were the ratios AB corresponding to the 6 groups of biomarkers (markers) in Table 1 to Table 6.

### 2. Experimental Results

The results were shown in Table 9 below.

TABLE 9

| | | Actual sample category | | | |
|---|---|---|---|---|---|
| | | | Renal | Urothelial | Prostate |
| | Test data set | Normal | cancer | cancer | cancer |
| Predicted | Normal | 23 | 6 | 2 | 4 |
| sample | Renal cancer | 3 | 5 | 1 | 5 |
| category | Urothelial cancer | 0 | 2 | 16 | 1 |
| | Prostate cancer | 3 | 5 | 2 | 4 |
| | Accuracy rate (%) | 79.3 | 27.8 | 76.2 | 28.6 |

| | |
|---|---|
| Total accuracy | 58.5 |
| rate (%) | |

The results showed that when the LASSO classification model was used, the accuracy rates of various predictions were lower than those of the integrated classification system (GUdetector) proposed by the present inventors, and the overall accuracy was only 58.5%.

### Comparative Example 2

### Using SVM Algorithm Model

### 1. Experimental Method

The method in the reference, CancerLocator: non-invasive cancer diagnosis and tissue-of-origin prediction using methylation profiles of cell to free DNA, was used.

The input data were the ratios AB corresponding to the 6 groups of biomarkers (markers) in Table 1 to Table 6.

### 2. Experimental Results

The results were shown in Table 10 below.

TABLE 10

| | | Actual sample category | | | |
|---|---|---|---|---|---|
| | | | Renal | Urothelial | Prostate |
| | Test data set | Normal | cancer | cancer | cancer |
| Predicted | Normal | 26 | 7 | 4 | 3 |
| sample | Renal cancer | 6 | 7 | 2 | 5 |
| category | Urothelial cancer | 3 | 2 | 18 | 3 |
| | Prostate cancer | 3 | 8 | 2 | 7 |
| | Accuracy rate (%) | 68.4 | 29.2 | 69.2 | 50.0 |

| | |
|---|---|
| Total accuracy | 54.7 |
| rate (%) | |

The results showed that when the SVM classification model was used, the accuracy rates of various predictions were lower than those of the integrated classification system (GUdetector) proposed by the present inventors, and the overall accuracy was only 54.7%.

### Comparative Example 3

### Random Forest Classification Model for Four Categories

### 1. Experimental Method

The method in the reference, Epigenetic profiling for the molecular classification of metastatic brain tumors, was used.

The input data were the ratios A/B corresponding to the 6 groups of biomarkers (markers) in Table 1 to Table 6.

### 2. Experimental Results

The results were shown in Table 11 below.

TABLE 11

| | | Actual sample category | | | |
|---|---|---|---|---|---|
| | | | Renal | Urothelial | Prostate |
| | Test data set | Normal | cancer | cancer | cancer |
| Predicted | Normal | 31 | 6 | 5 | 4 |
| sample | Renal cancer | 1 | 11 | 1 | 3 |
| category | Urothelial cancer | 2 | 1 | 18 | 2 |
| | Prostate cancer | 4 | 6 | 2 | 9 |
| | Accuracy rate (%) | 81.6 | 45.8 | 69.2 | 50.0 |

| | |
|---|---|
| Total accuracy | 65.1 |
| rate (%) | |

The results showed that when the random forest classification model for four categories was used, the accuracy rates of various predictions were lower than those of the integrated classification system (GUdetector) proposed by the present inventors, and the overall accuracy was only 65.1%.

Although the specific embodiments of the present invention have been described in detail, those skilled in the art will understand that according to all the teachings that have been disclosed, various modifications and substitutions can be made to those details, and these changes are all within the protection scope of the present invention. The full scope of the invention is given by the appended claims and any equivalents thereof.

1. A cfDNA classification method, comprising:
calculating a copy number variation data of cfDNA in a target sample;
calculating a similarity degree between the target cfDNA copy number variation data and the cfDNA copy number variation data of each category label; and
determining the category to which the target cfDNA belongs according to the similarity degree by using a classifier model.

2. The classification method according to claim 1, wherein determining the category to which the target cfDNA belongs comprises:
determining a correlation degree between the cfDNA copy number variation data of each category label and a human urogenital system tumor according to the similarity degree by using a random forest model;
determining the category to which the target cfDNA belongs according to the correlation degree by using the classifier model.

3. The classification method according to claim 2, wherein determining the correlation degree between the cfDNA copy number variation data of each category label and a human urogenital system tumor comprises:
sorting the cfDNA copy number variation data according to the correlation degree to form a vector sequence;
inputting the vector sequence into the random forest model, and determining the correlation degree between the cfDNA copy number variation data of the category label and the human urogenital system tumor.

4. The classification method according to claim 3, wherein the human urogenital system tumor is one or more selected from the group consisting of prostate cancer, urothelial cancer and renal cancer;
preferably, the renal cancer is clear renal cell carcinoma,
preferably, the urothelial cancer is upper urothelial cancer and/or bladder cancer,
preferably, the prostate cancer is prostate adenocarcinoma;
preferably, the human urogenital system tumor is diagnosed by tissue biopsy of a surgical sample.

5. The classification method according to claim 3, wherein the random forest model is at least 3 random forest binary classifiers, and is any one, two, three or four groups selected from the group consisting of the following Groups I to VI:
Group I.
normal-vs-renal cancer, normal-vs-urothelial cancer, normal-vs-prostate cancer;
Group II.
renal cancer-vs-normal, renal cancer-vs-urothelial cancer, renal cancer-vs-prostate cancer;
Group III.
urothelial cancer-vs-normal, urothelial cancer-vs-renal cancer, urothelial cancer-vs-prostate cancer;
Group IV.
prostate cancer-vs-normal, prostate cancer-vs-renal cancer, prostate cancer-vs-urothelial cancer.

6. The classification method according to claim 5, wherein each group is voted, the category corresponding to the group with the highest number of votes is the final category, and if there are groups with the same number of votes, the category corresponding to the group with the highest prediction probability in the groups with the same number of votes is the final category.

7. The classification method according to claim 1, wherein the copy number variation data of cfDNA in the target sample and/or the cfDNA copy number variation data of each category label is obtained by calculation from a sequencing data of cfDNA in a urine sample; preferably, the sequencing data is a whole-genome sequencing data; preferably, its sequencing depth is 1× to 5×.

8. The classification method according to claim 1, wherein the copy number variation data of cfDNA in the target sample and/or the cfDNA copy number variation data of each category label is calculated according to the following method:
dividing a genome of a sample to be tested into 5,000 to 500,000 bins with equal lengths or equal theoretical simulation copy numbers; normalizing the sequencing data, and calculating a ratio A/B of the number of reads corresponding to each bin,
wherein:
A represents the actual number of reads in a bin after GC content correction;
B represents the theoretical number of reads in the bin, which is obtained by dividing the total number of reads measured in the sample by the total number of bins;
the ratio A/B represents the copy number variation.

9. The classification method according to claim 8, wherein the genome of the sample to be tested is divided into 5,000 to 500,000 bins with equal lengths or equal theoretical simulation copy numbers by Varbin, CNVnator, ReadDepth or SegSeq;
and/or
calculating the ratio A/B of the number of reads corresponding to each bin by Varbin, CNVnator, ReadDepth or SegSeq.

10. The classification method according to claim 7, wherein the urine sample is a morning urine; preferably, the urine sample is a morning urine supernatant.

11. The classification method according to claim 8, wherein the ratio A/B is a ratio A/B of each biomarker in a biomarker combination,
wherein,
the biomarker combination comprises m biomarkers, and m represents a positive integer greater than or equal to 50;
the biomarker is a DNA fragment, correspondingly having an initiate site of A±n1 and a termination site of B±n2 on the chromosome;
wherein, the n1 and n2 are independently non-negative integers less than or equal to 60,000;
wherein, the chromosome, A and B are any one, any two, any three, any four, any five, any six or all seven groups selected from the group consisting of the following Groups (1) to (7);
(1) biomarkers for renal cancer vs. normal
TABLE 1

| No. | Chromosome | A | B |
|---|---|---|---|
| 1 | chr14 | 105173382 | 105228468 |
| 2 | chr4 | 126141989 | 126199070 |
| 3 | chr2 | 38340335 | 38396819 |
| 4 | chr4 | 120896519 | 120952988 |
| 5 | chr1 | 225263465 | 225322410 |
| 6 | chr3 | 49627990 | 49683004 |
| 7 | chr12 | 55710185 | 55770826 |
| 8 | chr2 | 198023323 | 198078345 |
| 9 | chr8 | 104278540 | 104334789 |
| 10 | chr15 | 102366051 | 102531392 |
| 11 | chr5 | 56684537 | 56739554 |
| 12 | chr12 | 2875899 | 2930969 |
| 13 | chr5 | 8084151 | 8143261 |
| 14 | chr13 | 24239617 | 24294704 |
| 15 | chr14 | 63064067 | 63121825 |
| 16 | chr10 | 32966493 | 33022298 |
| 17 | chr18 | 34499871 | 34555093 |
| 18 | chr18 | 27538044 | 27593083 |
| 19 | chr19 | 52518298 | 52574358 |
| 20 | chr3 | 148084127 | 148140439 |
| 21 | chr11 | 23395282 | 23450515 |
| 22 | chr19 | 53868391 | 53924718 |
| 23 | chr7 | 36856760 | 36911789 |
| 24 | chr19 | 55851675 | 55906675 |
| 25 | chr12 | 130622755 | 130677832 |
| 26 | chr8 | 88140900 | 88196181 |
| 27 | chr8 | 98015299 | 98073611 |
| 28 | chr22 | 24279186 | 24375790 |
| 29 | chr10 | 58285076 | 58342675 |
| 30 | chr1 | 193398457 | 193455292 |
| 31 | chr11 | 44170591 | 44225937 |
| 32 | chr3 | 99497035 | 99552049 |
| 33 | chr18 | 70229325 | 70284364 |
| 34 | chr3 | 86800483 | 86855497 |
| 35 | chr7 | 85391699 | 85446714 |
| 36 | chr2 | 222217699 | 222274614 |
| 37 | chr12 | 51953090 | 52017679 |
| 38 | chr2 | 231506603 | 231561625 |
| 39 | chr7 | 54479671 | 54534725 |
| 40 | chr5 | 40826473 | 40882045 |
| 41 | chr3 | 61041867 | 61097030 |
| 42 | chr1 | 71530378 | 71587704 |
| 43 | chr19 | 30375804 | 30434948 |
| 44 | chr5 | 103365336 | 103426037 |
| 45 | chr16 | 72331875 | 72390386 |
| 46 | chr12 | 77381964 | 77436979 |
| 47 | chr19 | 35419205 | 35474205 |
| 48 | chr8 | 131286269 | 131341291 |
| 49 | chr21 | 30776557 | 30834320 |
| 50 | chr9 | 17638202 | 17695124 |
| ; | | | |

(2) biomarkers for urothelial carcinoma vs. normal
TABLE 2

| No. | Chromosome | A | B |
|---|---|---|---|
| 1 | chr1 | 165542998 | 165598528 |
| 2 | chr20 | 45298182 | 45353725 |
| 3 | chr7 | 110250206 | 110305749 |
| 4 | chr8 | 34086369 | 34141392 |
| 5 | chr11 | 3080528 | 3135556 |
| 6 | chr8 | 81773551 | 81828573 |
| 7 | chr7 | 20604578 | 20660880 |
| 8 | chr8 | 101664207 | 101719230 |
| 9 | chr8 | 127300805 | 127363897 |
| 10 | chr3 | 175419548 | 175474633 |
| 11 | chr7 | 17433047 | 17488061 |
| 12 | chr11 | 126763962 | 126818990 |
| 13 | chr8 | 81328435 | 81383788 |
| 14 | chr1 | 160347268 | 160402416 |
| 15 | chr3 | 150917292 | 150976246 |
| 16 | chr8 | 78266536 | 78321853 |
| 17 | chr2 | 127233784 | 127288805 |
| 18 | chr9 | 119009696 | 119064910 |
| 19 | chr7 | 88363140 | 88418154 |
| 20 | chr6 | 168087004 | 168142398 |
| 21 | chr8 | 101056393 | 101111465 |
| 22 | chr9 | 121669613 | 121725772 |
| 23 | chr8 | 32804682 | 32859711 |
| 24 | chr1 | 160016845 | 160071870 |
| 25 | chr8 | 52860841 | 52916007 |
| 26 | chr1 | 184863212 | 184918237 |
| 27 | chr8 | 103059578 | 103114914 |
| 28 | chr11 | 131771420 | 131826541 |
| 29 | chr11 | 132772276 | 132827397 |
| 30 | chr8 | 142309304 | 142365059 |
| 31 | chr11 | 20866407 | 20922555 |
| 32 | chr9 | 9389289 | 9445177 |
| 33 | chr8 | 86975952 | 87030974 |
| 34 | chr8 | 68297698 | 68353353 |
| 35 | chr9 | 122009782 | 122064791 |
| 36 | chr8 | 61387868 | 61442890 |
| 37 | chr8 | 82499446 | 82554469 |
| 38 | chr9 | 118116705 | 118171814 |
| 39 | chr8 | 117772819 | 117827841 |
| 40 | chr9 | 135838140 | 135893149 |
| 41 | chr14 | 101522031 | 101577065 |
| 42 | chr8 | 81105039 | 81160812 |
| 43 | chr3 | 161042779 | 161098402 |
| 44 | chr9 | 104364444 | 104420690 |
| 45 | chr8 | 61111592 | 61166615 |
| 46 | chr20 | 31048866 | 31103880 |
| 47 | chr15 | 26890253 | 26945265 |
| 48 | chr4 | 28406811 | 28462319 |
| 49 | chr5 | 35031116 | 35086691 |
| 50 | chr10 | 101035266 | 101090283 |
| ; | | | |

(3) biomarkers for prostate cancer vs. normal
TABLE 3

| No. | Chromosome | A | B |
|---|---|---|---|
| 1 | chr6 | 150259849 | 150319419 |
| 2 | chr11 | 50065867 | 50143253 |
| 3 | chr2 | 223609354 | 223664376 |
| 4 | chr3 | 178315458 | 178370471 |
| 5 | chr5 | 142022744 | 142077815 |
| 6 | chr3 | 72366362 | 72421541 |
| 7 | chr14 | 51571751 | 51628678 |
| 8 | chr10 | 69911981 | 69966998 |
| 9 | chr9 | 75793867 | 75850925 |
| 10 | chr16 | 34486643 | 34542808 |
| 11 | chr16 | 75960918 | 76016022 |
| 12 | chr1 | 213593324 | 213648410 |
| 13 | chr14 | 81176000 | 81231314 |
| 14 | chr14 | 48680148 | 48735914 |
| 15 | chr1 | 66328295 | 66385662 |
| 16 | chr2 | 236695859 | 236750881 |
| 17 | chr16 | 34310644 | 34370518 |
| 18 | chr13 | 70644019 | 70699054 |
| 19 | chr1 | 104971030 | 105026648 |
| 20 | chr19 | 20033425 | 20088912 |
| 21 | chr12 | 41633765 | 41689196 |
| 22 | chr1 | 111186072 | 111241148 |
| 23 | chr11 | 81515081 | 81570551 |
| 24 | chr6 | 164934635 | 164990438 |
| 25 | chr7 | 88753879 | 88809024 |
| 26 | chr2 | 204421512 | 204476533 |
| 27 | chr13 | 38205109 | 38260137 |
| 28 | chr19 | 57310235 | 57365579 |
| 29 | chr5 | 172615261 | 172670278 |
| 30 | chr13 | 100608580 | 100663608 |
| 31 | chr1 | 248513391 | 248569321 |
| 32 | chr5 | 78269787 | 78325922 |
| 33 | chr10 | 12753021 | 12808156 |
| 34 | chr7 | 101911102 | 101966116 |
| 35 | chr17 | 30274080 | 30334227 |
| 36 | chr12 | 87935928 | 87995848 |
| 37 | chr9 | 12175965 | 12231559 |
| 38 | chr5 | 97385699 | 97441111 |
| 39 | chr8 | 3970051 | 4025074 |
| 40 | chr7 | 20604578 | 20660880 |
| 41 | chr8 | 32416104 | 32471278 |
| 42 | chr7 | 12021765 | 12077292 |
| 43 | chr20 | 11563548 | 11624648 |
| 44 | chr7 | 51785230 | 51840244 |
| 45 | chr19 | 16615231 | 16670336 |
| 46 | chr10 | 67343243 | 67399416 |
| 47 | chr11 | 10953369 | 11008630 |
| 48 | chr2 | 22332272 | 22390528 |
| 49 | chr17 | 10390372 | 10446415 |
| 50 | chr4 | 976667 | 1032082 |
| ; | | | |

(4) biomarkers for renal cancer vs. prostate cancer
TABLE 4

| No. | Chromosome | A | B |
|---|---|---|---|
| 1 | chr4 | 163059481 | 163114735 |
| 2 | chr4 | 6580383 | 6635407 |
| 3 | chr6 | 132270265 | 132325276 |
| 4 | chr2 | 82257259 | 82312280 |
| 5 | chr1 | 159394058 | 159452969 |
| 6 | chr9 | 105154079 | 105209849 |
| 7 | chr2 | 187699497 | 187754518 |
| 8 | chr4 | 126199070 | 126254087 |
| 9 | chr20 | 18854392 | 18909406 |
| 10 | chr7 | 15040427 | 15095480 |
| 11 | chr3 | 44690964 | 44747019 |
| 12 | chr11 | 57212694 | 57267722 |
| 13 | chr2 | 48829261 | 48885035 |
| 14 | chr12 | 133782920 | 133851895 |
| 15 | chr5 | 98900964 | 98963876 |
| 16 | chr11 | 86090264 | 86145292 |
| 17 | chr7 | 128477838 | 128533737 |
| 18 | chr2 | 32933311 | 32988604 |
| 19 | chr7 | 12693292 | 12748805 |
| 20 | chr4 | 95879059 | 95934075 |
| 21 | chr8 | 59989616 | 60044780 |
| 22 | chr12 | 32405135 | 32460143 |
| 23 | chr7 | 37972210 | 38027551 |
| 24 | chr11 | 128601685 | 128656714 |
| 25 | chr6 | 64185537 | 64240615 |
| 26 | chr7 | 107787926 | 107843035 |
| 27 | chr18 | 29036127 | 29091424 |
| 28 | chr16 | 47711531 | 47767836 |
| 29 | chr7 | 14590286 | 14645354 |
| 30 | chr11 | 55525982 | 55582014 |
| 31 | chr5 | 174061726 | 174116744 |
| 32 | chr14 | 44456533 | 44512749 |
| 33 | chr3 | 168694552 | 168750070 |
| 34 | chr4 | 114652704 | 114707721 |
| 35 | chr2 | 27431778 | 27486799 |
| 36 | chr4 | 107314339 | 107370716 |
| 37 | chr2 | 182718295 | 182773317 |
| 38 | chr10 | 19690582 | 19745774 |
| 39 | chr10 | 23594781 | 23649798 |
| 40 | chr3 | 3972580 | 4034015 |
| 41 | chr6 | 31323092 | 31379758 |
| 42 | chr8 | 128874896 | 128929933 |
| 43 | chr1 | 26256318 | 26311633 |
| 44 | chr5 | 161340570 | 161395587 |
| 45 | chr12 | 91346168 | 91401202 |
| 46 | chr19 | 2637431 | 2692582 |
| 47 | chr7 | 36856760 | 36911789 |
| 48 | chr9 | 27809024 | 27864032 |
| 49 | chr2 | 116615151 | 116670172 |
| 50 | chr9 | 112566383 | 112621994 |
| ; | | | |

(5) biomarkers for urothelial cancer vs. renal cancer
TABLE 5

| No. | Chromosome | A | B |
|---|---|---|---|
| 1 | chr4 | 163059481 | 163114735 |
| 2 | chr4 | 6580383 | 6635407 |
| 3 | chr6 | 132270265 | 132325276 |
| 4 | chr2 | 82257259 | 82312280 |
| 5 | chr1 | 159394058 | 159452969 |
| 6 | chr9 | 105154079 | 105209849 |
| 7 | chr2 | 187699497 | 187754518 |
| 8 | chr4 | 126199070 | 126254087 |
| 9 | chr20 | 18854392 | 18909406 |
| 10 | chr7 | 15040427 | 15095480 |
| 11 | chr3 | 44690964 | 44747019 |
| 12 | chr11 | 57212694 | 57267722 |
| 13 | chr2 | 48829261 | 48885035 |
| 14 | chr12 | 133782920 | 133851895 |
| 15 | chr5 | 98900964 | 98963876 |
| 16 | chr11 | 86090264 | 86145292 |
| 17 | chr7 | 128477838 | 128533737 |
| 18 | chr2 | 32933311 | 32988604 |
| 19 | chr7 | 12693292 | 12748805 |
| 20 | chr4 | 95879059 | 95934075 |
| 21 | chr8 | 59989616 | 60044780 |
| 22 | chr12 | 32405135 | 32460143 |
| 23 | chr7 | 37972210 | 38027551 |
| 24 | chr11 | 128601685 | 128656714 |
| 25 | chr6 | 64185537 | 64240615 |
| 26 | chr7 | 107787926 | 107843035 |
| 27 | chr18 | 29036127 | 29091424 |
| 28 | chr16 | 47711531 | 47767836 |
| 29 | chr7 | 14590286 | 14645354 |
| 30 | chr11 | 55525982 | 55582014 |
| 31 | chr5 | 174061726 | 174116744 |
| 32 | chr14 | 44456533 | 44512749 |
| 33 | chr3 | 168694552 | 168750070 |
| 34 | chr4 | 114652704 | 114707721 |
| 35 | chr2 | 27431778 | 27486799 |
| 36 | chr4 | 107314339 | 107370716 |
| 37 | chr2 | 182718295 | 182773317 |
| 38 | chr10 | 19690582 | 19745774 |
| 39 | chr10 | 23594781 | 23649798 |
| 40 | chr3 | 3972580 | 4034015 |
| 41 | chr6 | 31323092 | 31379758 |
| 42 | chr8 | 128874896 | 128929933 |
| 43 | chr1 | 26256318 | 26311633 |
| 44 | chr5 | 161340570 | 161395587 |
| 45 | chr12 | 91346168 | 91401202 |
| 46 | chr19 | 2637431 | 2692582 |
| 47 | chr7 | 36856760 | 36911789 |
| 48 | chr9 | 27809024 | 27864032 |
| 49 | chr2 | 116615151 | 116670172 |
| 50 | chr9 | 112566383 | 112621994 |
| ; | | | |

(6) biomarkers for urothelial cancer vs. prostate cancer
TABLE 6

| No. | Chromosome | A | B |
|---|---|---|---|
| 1 | chr3 | 88025277 | 88080310 |
| 2 | chr19 | 39394315 | 39449482 |
| 3 | chr20 | 31436554 | 31491568 |
| 4 | chr7 | 48432792 | 48487842 |
| 5 | chr8 | 87141019 | 87196120 |
| 6 | chr4 | 13859414 | 13914431 |
| 7 | chr1 | 160292243 | 160347268 |
| 8 | chr8 | 112245103 | 112300126 |
| 9 | chr8 | 11530043 | 11585066 |
| 10 | chr8 | 13932292 | 13987366 |
| 11 | chr3 | 152913886 | 152973883 |
| 12 | chr9 | 109516082 | 109571205 |
| 13 | chr11 | 8343925 | 8398954 |
| 14 | chr3 | 122030664 | 122085678 |
| 15 | chr5 | 87727661 | 87782722 |
| 16 | chr5 | 60881889 | 60936907 |
| 17 | chr14 | 40518423 | 40573582 |
| 18 | chr8 | 94667609 | 94724236 |
| 19 | chr8 | 101719230 | 101774274 |
| 20 | chr5 | 113527635 | 113584160 |
| 21 | chr3 | 103853900 | 103909150 |
| 22 | chr8 | 62393903 | 62449668 |
| 23 | chr8 | 124248002 | 124303024 |
| 24 | chr17 | 74131207 | 74186417 |
| 25 | chr14 | 52519339 | 52574927 |
| 26 | chr3 | 144795549 | 144851338 |
| 27 | chr3 | 84803116 | 84858323 |
| 28 | chr8 | 50523567 | 50578589 |
| 29 | chr8 | 88545977 | 88603606 |
| 30 | chr1 | 42119088 | 42174113 |
| 31 | chr20 | 43860121 | 43915135 |
| 32 | chr9 | 121061199 | 121116207 |
| 33 | chr9 | 118676908 | 118734641 |
| 34 | chr11 | 13163841 | 13219126 |
| 35 | chr11 | 57212694 | 57267722 |
| 36 | chr8 | 131892873 | 131948409 |
| 37 | chr11 | 16410024 | 16465871 |
| 38 | chr8 | 109405759 | 109460782 |
| 39 | chr5 | 158002797 | 158058189 |
| 40 | chr11 | 1579888 | 1635511 |
| 41 | chr8 | 51749113 | 51804136 |
| 42 | chr9 | 118562723 | 118621899 |
| 43 | chr17 | 29154317 | 29209332 |
| 44 | chr6 | 73471411 | 73528437 |
| 45 | chr3 | 87522168 | 87578480 |
| 46 | chr1 | 231915581 | 231971963 |
| 47 | chr8 | 117772819 | 117827841 |
| 48 | chr1 | 241691293 | 241746318 |
| 49 | chr9 | 92506773 | 92712072 |
| 50 | chr4 | 19120611 | 19176371 |
| ; | | | |

(7) biomarkers for normal vs. prostate cancer
TABLE 7

| No. | Chromosome | A | B |
|---|---|---|---|
| 1 | chr11 | 40374531 | 40429896 |
| 2 | chr12 | 61310253 | 61365625 |
| 3 | chr19 | 56809188 | 56866674 |
| 4 | chr2 | 145644444 | 145702420 |
| 5 | chr6 | 98011442 | 98066653 |
| 6 | chr7 | 88753879 | 88809024 |
| 7 | chr9 | 98761758 | 98817567 |
| 8 | chrY | 4474368 | 4588559 |
| 9 | chrY | 18884928 | 18940043 |
| 10 | chrY | 5632826 | 5746826 |
| 11 | chrY | 24371813 | 24427746 |
| 12 | chrY | 5948790 | 6035624 |
| 13 | chrY | 19228861 | 19283946 |
| 14 | chrY | 21484883 | 21542276 |
| 15 | chrY | 5746826 | 5851679 |
| 16 | chrY | 28707448 | 28764196 |
| 17 | chrY | 6599942 | 6664881 |
| 18 | chrY | 23799512 | 23860617 |
| 19 | chrY | 3427018 | 3545705 |
| 20 | chrY | 13573548 | 13635016 |
| 21 | chrY | 18387555 | 18551943 |
| 22 | chrY | 16529414 | 16585431 |
| 23 | chrY | 19111726 | 19166891 |
| 24 | chrY | 9020782 | 9081054 |
| 25 | chrY | 19451088 | 19508211 |
| 26 | chrY | 6720180 | 6778075 |
| 27 | chrY | 6349316 | 6458079 |
| 28 | chrY | 4163770 | 4261597 |
| 29 | chrY | 28648165 | 28707448 |
| 30 | chrY | 8741265 | 8796960 |
| 31 | chrY | 19283946 | 19339589 |
| 32 | chrY | 3970433 | 4073487 |
| 33 | chrY | 7346142 | 7402799 |
| 34 | chrY | 15149848 | 15205024 |
| 35 | chrY | 18774055 | 18829409 |
| 36 | chrY | 7290613 | 7346142 |
| 37 | chrY | 23743018 | 23799512 |
| 38 | chrY | 4700163 | 4811039 |
| 39 | chrY | 16473510 | 16529414 |
| 40 | chrY | 21654324 | 21709511 |
| 41 | chrY | 14418460 | 14477812 |
| 42 | chrY | 5851679 | 5948790 |
| 43 | chrY | 8685630 | 8741265 |
| 44 | chrY | 14650141 | 14705375 |
| 45 | chrY | 15605187 | 15663531 |
| 46 | chrY | 4073487 | 4163770 |
| 47 | chrY | 9399760 | 9457656 |
| 48 | chrY | 4366038 | 4474368 |
| 49 | chrY | 4937971 | 5066009 |
| 50 | chrY | 19564127 | 21039220 |

12. The classification method according to claim 11, wherein m is 50 to 300 or greater than 300, such as 50 to 100, 100 to 150, 150 to 200, 200 to 250, 250 to 300, 50, 100, 150, 200, 250 or 300.

13. The classification method according to claim 11, wherein n1 and n2 are independently 5,000, 4,000, 3,000, 2,000, 1500, 1,000, 500, 300, 200, 150, 100, 90, 80, 70, 60, 50, 40, 30, 20, 10, 5 or 0.

14. The classification method according to claim 11, wherein the biomarker is a cfDNA fragment; preferably, the cfDNA is derived from a human urine, particularly a human urine supernatant.

15. The classification method according to claim 11, wherein:
the chromosome, A and B are shown in any one, any two, any three, any four, any five, any six, or all seven groups selected from the group consisting of Groups (1) to (7).

16. A method for the detection, diagnosis, classification, disease risk assessment or prognosis assessment of a human urogenital system tumor, comprising the following steps (1), step (2), optionally step (3), and step (4):
(1) collecting a urine sample and extracting cfDNA;
(2) screening to obtain cfDNA fragments of 90 to 300 bp or cfDNA fragments of 100 to 300 bp,
(3) using the obtained cfDNA fragments to construct a whole genome library; and
(4) classifying the cfDNA fragments according to the classification method according to claim 1.

17. The method according to claim 16, wherein the urogenital system tumor is one or more selected from the group consisting of prostate cancer, urothelial cancer and renal cancer; preferably, the renal cancer is clean renal cell carcinoma, the urothelial cancer comprises upper urothelial cancer and bladder cancer, and the prostate cancer is prostate adenocarcinoma.

18. The method according to claim 16, wherein in step (1), the urine sample is a morning urine; preferably, the urine sample is a morning urine supernatant.

19. The method according to claim 16, wherein in step (2), the screening is screening by magnetic beads.

20. An apparatus for the detection, diagnosis, classification, disease risk assessment or prognosis assessment of a human urogenital system tumor, comprising:
I. ‘normal decision-making unit’:
normal-vs-renal cancer, normal-vs-urothelial cancer, normal-vs-prostate cancer;
II. ‘renal cancer decision-making unit’:
renal cancer-vs-normal, renal cancer-vs-urothelial cancer, renal cancer-vs-prostate cancer;
III. ‘urothelial cancer decision-making unit’:
urothelial cancer-vs-normal, urothelial cancer-vs-renal cancer, urothelial cancer-vs-prostate cancer; and
IV. ‘prostate cancer decision-making unit’:
prostate cancer-vs-normal, prostate cancer-vs-renal cancer, prostate cancer-vs-urothelial cancer.

21. An apparatus for the detection, diagnosis, classification, disease risk assessment or prognosis assessment of a human urogenital system tumor,
comprising a memory; and a processor coupled to the memory,
wherein,
the memory stores a program instruction to be executed by a processor, and the program instruction comprises any one, any two, any three, or all of four decision-making units selected from the group consisting of the following four decision-making units, wherein each decision-making unit comprises 3 random forest binary classifiers:
I. ‘normal decision-making unit’:
normal-vs-renal cancer, normal-vs-urothelial cancer, normal-vs-prostate cancer;
II. ‘renal cancer decision-making unit’:
renal cancer-vs-normal, renal cancer-vs-urothelial cancer, renal cancer-vs-prostate cancer;
III. ‘urothelial cancer decision-making unit’:
urothelial cancer-vs-normal, urothelial cancer-vs-renal cancer, urothelial cancer-vs-prostate cancer;
IV. ‘prostate cancer decision-making unit’:
prostate cancer-vs-normal, prostate cancer-vs-renal cancer, prostate cancer-vs-urothelial cancer.

22. The apparatus according to claim 21, wherein the processor is configured to execute a cfDNA classification method based on instruction stored in the memory device, wherein the cfDNA classification method comprises:
calculating a copy number variation data of cfDNA in a target sample;
calculating a similarity degree between the target cfDNA copy number variation data and the cfDNA copy number variation data of each category label; and
determining the category to which the target cfDNA belongs according to the similarity degree by using a classifier model.

23. The apparatus according to claim 11, wherein the urogenital system tumor is one or more selected from the group consisting of prostate cancer, urothelial cancer and renal cancer;
preferably, the renal cancer is clear renal cell carcinoma,
preferably, the urothelial cancer is upper urothelial cancer and/or bladder cancer,
preferably, the prostate cancer is prostate adenocarcinoma.

24-25. (canceled)

26. A biomarker combination, which is a combination of the biomarkers according to claim 11.
